(19) United States
(12) Patent Application Publication
(10) Pub. No.: US 2007/0238671 A1
(43) Pub. Date: Oct. 11, 2007

(54) Isopeptide Gap Junction Modulators

(76) Inventor: Bjarne Due Larsen, Roskilde (DK)

Correspondence Address: KIRKPATRICK & LOCKHART PRESTON GATES ELLIS LLP;(FORMERLY KIRKPATRICK&LOCKHART NICHOLSON GRAHAM), STATE STREET FINANCIAL CENTER, ONE LINCOLN STREET, BOSTON, MA, 02111-2950, US

(21) Appl. No.: 10/584,373

(22) PCT Filed: Dec. 23, 2004
(86) PCT No.: PCT/GB04/05416

§ 371 Date: Jun. 1, 2007

Related U.S. Application Data
(60) Provisional application No. 60/532,111, filed on Dec. 23, 2003.

(30) Foreign Application Priority Data
Dec. 23, 2003 (DK) 2003 01928

(51) Int. Cl. A61K 38/00; A61K 38/04; A61P 9/06; C07C 233/00; C07K 5/00

(52) U.S. Cl. 514/19; 530/300

(57) ABSTRACT

The invention relates to isopeptides capable of modulating intracellular gap junctional communication. The invention further relates to methods of using the isopeptides to maintain or enhance such communication. In one aspect, the isopeptides are antiarrhythmic isopeptides which target the same cells targeted by AAP, AAP10, HP5, and/or functional analogs thereof, i.e. the isopeptides are able to modulate the function of these cells by agonizing or antagonizing the function of AAP, AAP10, HP5, and/or functional analogs thereof.

### FIELD OF THE INVENTION

The invention relates to isopeptides capable of modulating intracellular gap junctional communication. The invention further relates to methods of using the isopeptides to maintain or enhance such communication.

### BACKGROUND

There is increasing recognition that intercellular communication is essential for cellular homeostasis, proliferation and differentiation. Such communication is believed to be facilitated by gap junctions. These structures are thought to be a route for coupling cells and permiting “cross-talk”. See generally, Sperelakis, N., (1989) Cell Interactions and Gap Junctions by N. Sperelakis, William C. Cole (Editor).

There have been efforts to understand the structure and function of gap junctions. For instance, there have been reports that such junctions are a type of complex formed between adjacent cells. Most gap junctions are thought to consist of aggregated channels that directly link the interiors (cytoplasms) of neighboring cells. In adult mammals, gap junctions are found in most cell types with the exception of circulating blood elements.

More specifically, there is acknowledgement that gap junctions are specialized regions of the cell membrane with clusters of hundreds to thousands of densely packed gap junction channels (comprising two hemichannels or connexins). Many are thought to directly connect the cytoplasmic compartments of two neighboring cells. The gap junction channel can switch between an open and a closed state. In the open state ions and small molecules are thought to pass through the pore of the gap junction channel. The conduction of electrical impulses and intercellular diffusion of signaling molecules take place through the gap junctions.

The “cross-talk” between gap junctions has been referred to as gap junctional intracellular communication (GJIC). It is thought to play an important role in the regulation of cell metabolism, proliferation, cell-to-cell signaling, and tissue integrity.

For instance, GJIC is thought to permit rapid equilibration of nutrients, ions, and fluids between cells. Gap junctions are also thought to serve as electrical synapses in electrically excitable cells. In many tissues, electrical coupling is thought to permit more rapid cell-to-cell transmission of action potentials than chemical synapses. In cardiomyocytes and smooth muscle cells, for instance, this is thought to assist synchronous contraction.

There have been reports of other functions mediated by GJIC. For example, GJIC is thought to enhance the responsiveness of tissues to external stimuli. Second messengers are generally believed to be small enough to pass from hormonally activated cells to quiescent cells through junctional channels and activate the latter.

Additionally, there have been reports that gap junctions may provide intercellular pathways for chemical and/or electrical developmental signals and assist in defining the boundaries of developmental compartments. It has been disclosed that GJIC occurs in specific patterns in embryonic cells and the impairment of GJIC has been related to developmental anomalies and the teratogenic effects of many chemicals. Further, GJIC is thought to assist co-ordination of cell activities.

Some reports have established a link between abnormalities in GJIC and a range of disease states has been established both in vitro and in vivo. For example, there is thought to be a link between abnormalities in connexins and heart disease. Several studies of the expression and distribution of Cx43 in hearts describe a reduced degree of Cx43 expression and a changed pattern of distribution for this gap junction protein. See Kaprielian, R. R., et al. (1998) Circulation 97: 651-660; and Saffitz, J. E., et al., (1999) Cardiovasc Res. 42: 309-317.

Accordingly, there is recognition in the field of relationship between a malfunction or absence of gap junctions and an increased risk of arrhythmias. There is thought to be a further relationship between altered connexin expression/distribution and chronic heart disease.

There is increasing understanding that many of the antiarrhythmic peptides positively impact GJIC, often without affecting action potential duration or shape. Further, many of such peptides are thought to lack undesirable proarrhythmic side-effects. Such effects are thought to limit use of many currently available antiarrhythmic drugs. Moreover, AAP, as well as certain AAP derivatives, are thought to have some undesired features, e.g., low stability and a need for high doses before therapeutic efficacy is achieved.

It would be desirable to have effective isopeptide modulators of GJIC. The present invention discloses such isopeptides.

### SUMMARY OF THE INVENTION

The invention generally relates to isopeptides that modulate gap junction intercellular communication (GJIC). The invention has a wide spectrum of useful applications including use in the treatment or prevention of pathologies associated with impaired GJIC.

A preferred isopeptide in accord with the present invention is represented by the following general formula (I):

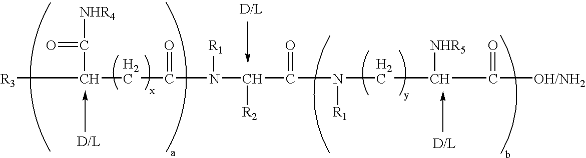

where, if a is 1 then b is 0;

if a is 0 then b is 1;

x and y independently is 1-7;

R₁ is H or CH₃, preferably H.

R₂ is a side chain of any amino acid: e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and sarcosine. Preferably R₂ is a side chain of alanine or glycine. Preferably, R₂ is the side chain of the amino acid glycine when a is 0 and b is 1, or alanine when a is 1 and b is 0;

R₃ is selected from the group consisting of H, NH₂, NHR, NR₂, ⁺NR₃, OH, SH, RO, RS, RSO, RSO₂, COR, CSR, COOH, COOR, CONH₂, CONHR, CONR₂, OCOR, SCOR, (R=alkyl, alkenyl, aryl, aralkyl, cycloalkyl, etc.). Preferably R₃ is H or NH₂; and

R₄ and R₅ independently comprise a hydrophobic group, and preferably, comprise an aromatic carbon ring, more preferably, comprising a 6- or 12-membered aromatic carbon ring. In one aspect, the aromatic ring is substituted with at least one of: a lower alkyl, alkoxy, hydroxyl, carboxy, amine, thiol, hydrazide, amide, halide, hydroxyl, ether, amine, nitrile, imine, nitro, sulfide, sulfoxide, sulfone, thiol, aldehyde, keto, carboxy, ester, an amide group; including seleno and thio derivatives thereof. Optionally substituted rings also include sulfide, sulfoxide, sulfone, and thiol derivates with, or without, a seleno group. Preferably the aromatic ring is substituted with at least one of: a lower alkyl, alkoxy, halide, nitrile and nitro group. More preferably, the aromatic ring is substituted with at least one of an alkoxy or nitro group. A lower alkyl group is preferably a methyl group. An alkoxy group is preferably a methoxy group. A halide group is preferably a chloride or fluoride group. A nitrile group is preferably a cyano group. In embodiments in which the aromatic carbon ring is substituted, such substitutions will typically number less than about 10 substitutions, more preferably, substitutions will number about 1 to to 5, or 1 to 2 substitutions.

The aromatic carbon ring may be selected from a benzyl, phenyl and naphthyl group, and is preferably a benzyl group.

Preferably, R₄ and R₅ independently comprise a benzyl group substituted with at least one of a lower alkyl, alkoxy, halide, nitrile or nitro group. More preferably, R₄ and R₅ independently comprise a benzyl group substituted with at least one of a nitro or alkoxy group. A lower alkyl group is preferably a methyl group, an alkoxy group is preferably a methoxy group, a halide group is preferably a chloride or fluoride group, and a nitrile group is preferably a cyano group.

Isopeptides of the invention may be represented by Formula I, where R₁ is H, R₂ is a side chain of an amino acid alanine or glycine, R₃ is H or NH₂, and R₄ and R₅ comprise a benzyl group substituted with at least one of a nitro or methoxy group.

Preferably, when R₂ is the side chain of amino acid glycine, a is 0, b is 1 and R₁ is H.

Preferably, when R₂ is the side chain of amino acid alanine, a is 1, b is 0 and R₁ is H.

Preferably, when a is 0 and b is 1, y is 4 and R₁ is H. More preferably, when a is 0 and b is 1, y is 4, R₁ is H and R₅ comprises a benzyl group. Preferably, when a is 1 and b is 0, x is 1 or 2 and R₁ is H. More preferably, when a is 1 and b is 0, x is 1 or 2, R₁ is H and R₄ comprises a benzyl group.

When R₂ is the side chain of amino acid glycine and a is 0, b is 1, preferably y is 4 and R₁ is H. When R₂ is the side chain of amino acid alanine and a is 1, b is 0, preferably x is 1 or 2 and R₁ is H.

Preferred isopeptides may be represented by Formula I where R₁ is H; R₂ is the side chain of the amino acid glycine when a is 0, b is 1 and y is 4, or alanine when a is 1, b is 0 and x is 1 or 2; R₃ is H or NH₂; and R₄ and R₅ comprise a benzyl group. Further, the benzyl group is preferably substituted with a nitro or methoxy group.

Such isopeptides may also comprise a peptide bond that is alkylated or otherwise modified to stabilize the isopeptide against enzymatic degradation and/or may comprise D-amino acids, isoforms of amino acids or a combination of D-amino acids and isoforms of amino acids.

In another embodiment the invention concerns more specific isopeptides represented by the following general Formula (II):

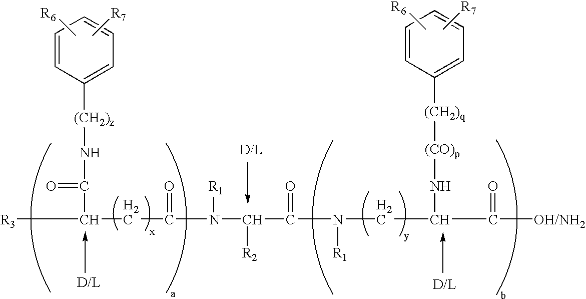

where, if a=1 then b=0;

if a=0 then b=1;

x and y independently=1-7;

z=1-6;

q=0-6;

p=0-1

R₁═H or CH₃, preferably H.

R₂=side chain of any amino acid: e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and sarcosine. Preferably R₂ is a side chain of alanine or glycine. Preferably, R₂ is the side chain of the amino acid glycine when a is 0 and b is 1, or alanine when a is 1 and b is 0;

R₃ is selected from the group consisting of H, NH₂, NHR, NR₂, ⁺NR₃, OH, SH, RO, RS, RSO, RSO₂, COR, CSR, COOH, COOR, CONH₂, CONHR, CONR₂, OCOR, and SCOR, wherein R=alkyl, alkenyl, aryl, aralkyl, or cycloalkyl. Preferably R₃ is H or NH₂; and

R₆ and R₇ are independently selected from the group consisting of H, alkyl, alkenyl, aryl, aralkyl, halogen, CN, NO₂, alkoxy, aryloxy, aralkyloxy, thioalkoxy, thioaryloxy, thioaralkyloxy, +S(CH₃)₂, SO₃H, SO₂R, NH₂, NHR, NR₂, ⁺NR₃, OH, SH, COOH, COOR, CONH₂, CONHR, CONR₂, CH₂OH, NCO, NCOR, NHOH, NHNH₂, NHNRH, CH₂OCOR, CH₂OCSR, COR, CSR, CSOR, CF₃, and CCl₃, and wherein R is alkyl, alkenyl, aryl, aralkyl, or cycloalkyl. Preferably, R₆ and R₇ are independently selected from the group consisting of H, alkyl, halogen, CN, NO₂, alkoxy, CF₃. More preferably, R₆ and R₇ are independently selected from the group consisting of H, NO₂ and alkoxy. An alkyl is preferably methyl, a halogen is preferably chlorine or fluorine, and an alkoxy is preferably methoxy.

Isopeptides of the invention may be represented by Formula II, where R₁ is H, R₂ is a side chain of an amino acid alanine or glycine, R₃ is H or NH₂, and R₆ and R₇ are independently selected from the group consisting of H, NO₂ and methoxy.

Preferably, when R₂ is the side chain of amino acid glycine, a is 0, b is 1 and R₁ is H.

Preferably, when R₂ is the side chain of amino acid alanine, a is 1, b is 0 and R₁ is H.

Preferably, when a is 0 and b is 1, y is 4 and R₁ is H. More preferably, when a is 0 and b is 1, y is 4, p is 1, q is 0, and R₁ is H. Preferably, when a is 1 and b is 0, x is 1 or 2 and R₁ is H. More preferably, when a is 1 and b is 0, x is 1 or 2, z is 1, and R₁ is H.

When R₂ is the side chain of amino acid glycine and a is 0, b is 1, preferably y is 4 and R₁ is H. When R₂ is the side chain of amino acid alanine and a is 1, b is 0, preferably x is 1 or 2 and R₁ is H.

Preferred isopeptides may be represented by Formula II where R₁ is H; R₂ is the side chain of the amino acid glycine when a is 0, b is 1 and y is 4, p is 1, q is 0, or alanine when a is 1, b is 0 and x is 1 or 2, z is 1; R₃ is H or NH₂. Further, R₆ and R₇ are independently selected from the group consisting of H, NO₂ and methoxy.

The aromatic carbon ring preferably comprises a substituent at the 4-position. The substituent may be any group listed herein as one which may be a substituent of an aromatic ring.

Preferably, the substituent is selected from the group consisting of: a lower alkyl, alkoxy, halide, nitrile and nitro group. Preferably the substituent is a nitro or alkoxy group. An alkoxy group is preferably a methoxy group. In Formula II, when R₆ or R₇ is H, R₇ or R₆, respectively, may be a nitro or methoxy group.

Isopeptides of the invention may be represented by general Formula II. Isopeptides of the invention may have the general form:
H—first amino acid moiety—second amino acid moiety —OH.

An “amino acid moiety” is defined as a moiety comprising an amino acid. An amino acid may be any amino acid, e.g. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and sarcosine. Such a moiety may additionally comprise a further chemical group or groups, for example a hydrophobic group, for example an aromatic carbon ring, for example, a 6-membered aromatic carbon ring. Included within the definition is a moiety which does not comprise any further chemical groups, such that it consists of an amino acid, i.e. the amino acid moiety comprises an amino acid only, without the addition of further chemical groups.

An amino acid moiety may comprise an amino acid selected from the group consisting of glycine, asparagine, glutamine, lysine, alanine and sarcosine. An amino acid moiety may comprise an amino acid selected from the group consisting of glycine, asparagine, glutamine, lysine and alanine. An amino acid moiety may be selected from the group consisting of glycine, glutamine, lysine and alanine.

A first amino acid moiety may comprise an amino acid selected from the group consisting of glycine, asparagine and glutamine. A second amino acid moiety may be selected from the group consisting of lysine, alanine and sarcosine. Preferably, an amino acid of the first amino acid moiety is glycine or glutamine. Preferably, an amino acid of the second amino acid moiety is lysine or alanine.

A first or second amino acid moiety comprising an amino acid may additionally comprise a hydrophobic group, for example an aromatic carbon ring, for example, a 6- or 12-membered aromatic carbon ring. The aromatic ring may be selected from a benzyl, benzoyl, phenyl or naphthyl group. Preferably, the aromatic ring is a benzyl or benzoyl group. Preferably, either the first amino acid moiety or the second amino acid moiety comprises an aromatic ring. Where a first amino acid moiety comprises an aromatic ring, the aromatic ring is preferably a benzyl group. Where a second amino acid moiety comprises an aromatic ring, the aromatic ring is preferably a benzoyl group.

An aromatic ring may be substituted with at least one of: a lower alkyl, alkoxy, hydroxyl, carboxy, amine, thiol, hydrazide, amide, halide, hydroxyl, ether, amine, nitrile, imine, nitro, sulfide, sulfoxide, sulfone, thiol, aldehyde, keto, carboxy, ester, an amide group; including seleno and thio derivatives thereof. Optionally substituted rings also include sulfide, sulfoxide, sulfone, and thiol derivates with, or without, a seleno group. Preferably, an aromatic ring may be substituted with at least one of a lower alkyl, alkoxy, halide, nitrile and nitro group, or may be unsubstituted. More preferably, an aromatic ring is substituted with at least one of a nitro and alkoxy group. A lower alkyl group is preferably a methyl group. An alkoxy group is preferably a methoxy group. A halide group is preferably a chloride or fluoride group. A nitrile group is preferably a cyano group.

An aromatic ring may be substituted with at least one of: H, alkyl, alkenyl, aryl, aralkyl, halogen, CN, NO₂, alkoxy, aryloxy, aralkyloxy, thioalkoxy, thioaryloxy, thioaralkyloxy, +S(CH₃)₂, SO₃H, SO₂R, NH₂, NHR, NR₂, ⁺NR₃, OH, SH, COOH, COOR, CONH₂, CONHR, CONR₂, CH₂OH, NCO, NCOR, NHOH, NHNH₂, NHNRH, CH₂OCOR, CH₂OCSR, COR, CSR, CSOR, CF₃, and CCl₃, and wherein R is alkyl, alkenyl, aryl, aralkyl, and cycloalkyl. Preferably, an aromatic ring may be substituted with at least one of: alkyl, halogen, CN, NO₂, alkoxy and CF₃, or may be unsubstituted. More preferably, an aromatic ring may be substituted with at least one of: NO₂ and alkoxy. An alkyl is preferably methyl, a halogen is preferably chlorine or fluorine, and an alkoxy is preferably methoxy. Preferably, an aromatic ring may be substituted with at least one of: a methyl, chloro, fluoro, trifluoromethyl, cyano, nitro and methoxy group, or may be unsubstituted. More preferably, an aromatic ring may be substituted with at least one of a nitro and methoxy group.

Preferably, an aromatic ring is substituted at the 4-position.

Such isopeptides may comprise a peptide bond that is alkylated or otherwise modified to stabilize the isopeptide against enzymatic degradation and/or may comprise D-amino acids, isoforms of amino acids or a combination of D-amino acids and isoforms of amino acids.

The first amino acid moiety may comprise glycine or may comprise glutamine and an aromatic ring. The second amino acid moiety may comprise alanine or may comprise lysine and an aromatic ring. Preferably, the first amino acid moiety may comprise glycine or may comprise glutamine and an aromatic ring, and the second amino acid moiety may comprise alanine or may comprise lysine and an aromatic ring. More preferably, the first amino acid moiety may comprise glycine and the second amino acid moiety may comprise lysine and an aromatic ring, or the first amino acid moiety may comprise glutamine and an aromatic ring and the second amino acid moiety may comprise alanine.

Where the first amino acid moiety comprises an aromatic ring, the aromatic ring is preferably substituted with a nitro or methoxy group, more preferably a methoxy group. Where the second amino acid moiety comprises an aromatic ring, the aromatic ring is preferably substituted with a nitro or methoxy group.

The isopeptides according to the invention have a wide variety of important uses and advantages.

For instance, the present isopeptides may be used for preventing or treating conditions associated with impaired gap junction function resulting in reduced intercellular communication or misregulated cellular communication. In one aspect, the invention provides a method of administering to an individual having, or at risk of developing such a condition, a therapeutically effective amount of any of the isopeptides described above. Preferably, administration is oral. In one preferred aspect, an individual is a human being. Preferably, the isopeptide is selected from the group consisting of the isopeptides shown in Table 2.

Examples of conditions which can be treated include, but are not limited to, cardiovascular disease, inflammation of airway epithelium, disorders of alveolar tissue, bladder incontinence, impaired hearing due to diseases of the cochlea, endothelial lesions, diabetic retinopathy and diabetic neuropathy, ischemia of the central nervous system and spinal cord, dental tissue disorders including periodontal disease, kidney diseases, failures of bone marrow transplantation, wounds, erectile dysfunction, urinary bladder incontinence, neuropathic pain, subchronic and chronic inflammation, cancer and failures of bone marrow and stem cell transplantation, conditions which arise during transplantation of cells and tissues or during medical procedures such as surgery; as well as conditions caused by an excess of reactive oxygen species and/or free radicals and/or nitric oxide.

The invention additionally provides pharmaceutical compositions suitable for use in the methods described above, comprising any of the isopeptides described above and a pharmaceutically acceptable carrier. Preferably, the carrier is sterile, pyrogen-free and virus-free. Further, the invention is concerned with the use of the present isopeptides for the manufacture of a medicament for the treatment of the above medical indications. Preferably, the isopeptide is selected from the group consisting of the isopeptides shown in Table 2.

### BRIEF DESCRIPTION OF THE DRAWINGS

FIG. 1A+1B show exemplary synthesis schemes for generating isopeptides according to formula (I).

FIG. 2A+B show exemplary synthesis schemes for generating isopeptides according to formula (II).

### DETAILED DESCRIPTION OF THE INVENTION

As discussed, the invention relates to isopeptides that modulate gap junction intercellular communication (GJIC). The invention has a wide spectrum of useful applications including use in the treatment or prevention of pathologies associated with impaired GJIC. Particular invention isopeptides are represented by Formula I above.

More specific isopeptides according to the invention are represented by the following general Formula II:

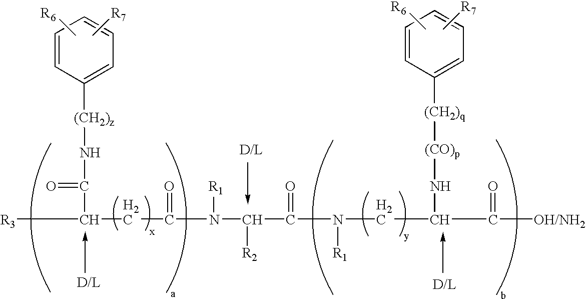

where, if a=1 then b=0;

if a=0 then b=1;

x and y independently=1-7;

z=1-6;

q=0-6;

p=0-1

R₁═H or CH₃, preferably H.

R₂=side chain of any amino acid: e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and sarcosine. Preferably R₂ is a side chain of alanine or glycine. Preferably, R₂ is the side chain of the amino acid glycine when a is 0 and b is 1, or alanine when a is 1 and b is 0;

R₃ is selected from the group consisting of H, NH₂, NHR, NR₂, ⁺NR₃, OH, SH, RO, RS, RSO, RSO₂, COR, CSR, COOH, COOR, CONH₂, CONHR, CONR₂, OCOR, and SCOR, wherein R=alkyl, alkenyl, aryl, aralkyl, or cycloalkyl. Preferably R₃ is H or NH₂; and

R₆ and R₇ are independently selected from the group consisting of H, alkyl, alkenyl, aryl, aralkyl, halogen, CN, NO₂, alkoxy, aryloxy, aralkyloxy, thioalkoxy, thioaryloxy, thioaralkyloxy, +S(CH₃)₂, SO₃H, SO₂R, NH₂, NHR, NR₂, ⁺NR₃, OH, SH, COOH, COOR, CONH₂, CONHR, CONR₂, CH₂OH, NCO, NCOR, NHOH, NHNH₂, NHNRH, CH₂OCOR, CH₂OCSR, COR, CSR, CSOR, CF₃, and CCl₃, and wherein R is alkyl, alkenyl, aryl, aralkyl, or cycloalkyl. Preferably, R₆ and R₇ are independently selected from the group consisting of H, alkyl, halogen, CN, NO₂, alkoxy, CF₃. More preferably, R₆ and R₇ are independently selected from the group consisting of H, NO₂ and alkoxy. An alkyl is preferably methyl, a halogen is preferably chlorine or fluorine, and an alkoxy is preferably methoxy.

Isopeptides of the invention may be represented by Formula II, where R₁ is H, R₂ is a side chain of an amino acid alanine or glycine, R₃ is H or NH₂, and R₆ and R₇ are independently selected from the group consisting of H, NO₂ and methoxy.

Preferably, when R₂ is the side chain of amino acid glycine, a is 0, b is 1 and R₁ is H.

Preferably, when R₂ is the side chain of amino acid alanine, a is 1, b is 0 and R₁ is H.

Preferably, when a is 0 and b is 1, y is 4 and R₁ is H. More preferably, when a is 0 and b is 1, y is 4, p is 1, q is 0, and R₁ is H. Preferably, when a is 1 and b is 0, x is 1 or 2 and R₁ is H. More preferably, when a is 1 and b is 0, x is 1 or 2, z is 1, and R₁ is H.

When R₂ is the side chain of amino acid glycine and a is 0, b is 1, preferably y is 4 and R₁ is H.

When R₂ is the side chain of amino acid alanine and a is 1, b is 0, preferably x is 1 or 2 and R₁ is H.

Preferred isopeptides may be represented by Formula II where R₁ is H; R₂ is the side chain of the amino acid glycine when a is 0, b is 1 and y is 4, p is 1, q is 0, or alanine when a is 1, b is 0 and x is 1 or 2, z is 1; R₃ is H or NH₂. Further, R₆ and R₇ are independently selected from the group consisting of H, NO₂ and methoxy.

The aromatic carbon ring preferably comprises a substituent at the 4-position. The substituent may be any group listed herein as one which may be a substituent of an aromatic ring. Preferably, the substituent is selected from the group consisting of: a lower alkyl, alkoxy, halide, nitrile and nitro group. Preferably the substituent is a nitro or alkoxy group. An alkoxy group is preferably a methoxy group. In Formula II, when R₆ or R₇ is H, R₇ or R₆, respectively, may be a nitro or methoxy group.

Particularly preferred isopeptides of the invention may be represented by Formula II where R₁ is H; R₂ is the side chain of the amino acid glycine; a is 0, b is 1 and y is 4, p is 1, q is 0; R₃ is H or NH₂; and R₆ or R₇ is H and R₇ or R₆, respectively, is a nitro or methoxy group. Particularly preferred isopeptides of the invention may be represented by Formula II where R₁ is H; R₂ is the side chain of the amino acid alanine; a is 1, b is 0 and x is 1 or 2, z is 1; R₃ is H or NH₂; and R₆ or R₇ is H and R₇ or R₆, respectively, is a methoxy group.

Isopeptides of the invention may be represented by general Formula II. Isopeptides of the invention may have the general form:
H—first amino acid moiety—second amino acid moiety —OH.

An “amino acid moiety” is defined as a moiety comprising an amino acid. An amino acid may be any amino acid, e.g. alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and sarcosine. Such a moiety may additionally comprise a further chemical group or groups, for example a hydrophobic group, for example an aromatic carbon ring, for example, a 6- or 12-membered aromatic carbon ring. Included within the definition is a moiety which does not comprise any further chemical groups such that it consists of an amino acid, i.e. the amino acid moiety comprises an amino acid alone, without the addition of further chemical groups.

An amino acid moiety may comprise an amino acid selected from the group consisting of glycine, asparagine, glutamine, lysine, alanine and sarcosine. An amino acid moiety may comprise an amino acid selected from the group consisting of glycine, asparagine, glutamine, lysine and alanine. An amino acid moiety may be selected from the group consisting of glycine, glutamine, lysine and alanine.

A first amino acid moiety may comprise an amino acid selected from the group consisting of glycine, asparagine and glutamine. A second amino acid moiety may be selected from the group consisting of lysine, alanine and sarcosine. Preferably, an amino acid of the first amino acid moiety is glycine or glutamine. Preferably, an amino acid of the second amino acid moiety is lysine or alanine.

A first or second amino acid moiety comprising an amino acid may additionally comprise a hydrophobic group, for example an aromatic carbon ring, for example, a 6- or 1 2-membered aromatic carbon ring. The aromatic ring may be selected from a benzyl, benzoyl, phenyl or naphthyl group. Preferably, the aromatic ring is a benzyl or benzoyl group. Preferably, either the first amino acid moiety or the second amino acid moiety comprises an aromatic ring. Where a first amino acid moiety comprises an aromatic ring, the aromatic ring is preferably a benzyl group. Where a second amino acid moiety comprises an aromatic ring, the aromatic ring is preferably a benzoyl group.

An aromatic ring may be substituted with at least one of: a lower alkyl, alkoxy, hydroxyl, carboxy, amine, thiol, hydrazide, amide, halide, hydroxyl, ether, amine, nitrile, imine, nitro, sulfide, sulfoxide, sulfone, thiol, aldehyde, keto, carboxy, ester, an amide group; including seleno and thio derivatives thereof. Optionally substituted rings also include sulfide, sulfoxide, sulfone, and thiol derivates with, or without, a seleno group. Preferably, an aromatic ring may be substituted with at least one of a lower alkyl, alkoxy, halide, nitrile and nitro group, or may be unsubstituted. More preferably, an aromatic ring is substituted with at least one of a nitro and alkoxy group. A lower alkyl group is preferably a methyl group. An alkoxy group is preferably a methoxy group. A halide group is preferably a chloride or fluoride group. A nitrile group is preferably a cyano group.

An aromatic ring may be substituted with at least one of: H, alkyl, alkenyl, aryl, aralkyl, halogen, CN, NO₂, alkoxy, aryloxy, aralkyloxy, thioalkoxy, thioaryloxy, thioaralkyloxy, +S(CH₃)₂, SO₃H, SO₂R, NH₂, NHR, NR₂, ⁺NR₃, OH, SH, COOH, COOR, CONH₂, CONHR, CONR₂, CH₂OH, NCO, NCOR, NHOH, NHNH₂, NHNRH, CH₂OCOR, CH₂OCSR, COR, CSR, CSOR, CF₃, and CCl₃, and wherein R is alkyl, alkenyl, aryl, aralkyl, and cycloalkyl. Preferably, an aromatic ring may be substituted with at least one of: alkyl, halogen, CN, NO₂, alkoxy and CF₃, or may be unsubstituted. More preferably, an aromatic ring may be substituted with at least one of: NO₂ and alkoxy. An alkyl is preferably methyl, a halogen is preferably chlorine or fluorine, and an alkoxy is preferably methoxy. Preferably, an aromatic ring may be substituted with at least one of: a methyl, chloro, fluoro, trifluoromethyl, cyano, nitro and methoxy group, or may be unsubstituted. More preferably, an aromatic ring may be substituted with at least one of a nitro and methoxy group.

Preferably, an aromatic ring is substituted at the 4-position.

Such isopeptides may comprise a peptide bond that is alkylated or otherwise modified to stabilize the isopeptide against enzymatic degradation and/or may comprise D-amino acids, isoforms of amino acids or a combination of D-amino acids and isoforms of amino acids.

The first amino acid moiety may comprise glycine, or may comprise glutamine and an aromatic ring. The second amino acid moiety may comprise alanine, or may comprise lysine and an aromatic ring. Preferably, the first amino acid moiety comprises glycine, or comprises glutamine and an aromatic ring, and the second amino acid moiety comprises alanine, or comprises lysine and an aromatic ring. More preferably, the first amino acid moiety comprises glycine and the second amino acid moiety comprises lysine and an aromatic ring, or the first amino acid moiety comprises glutamine and an aromatic ring and the second amino acid moiety comprises alanine.

Where the first amino acid moiety comprises an aromatic ring, the aromatic ring is preferably substituted with a nitro or methoxy group, more preferably a methoxy group. Where the second amino acid moiety comprises an aromatic ring, the aromatic ring is preferably substituted with a nitro or methoxy group.

Preferred isopeptides may be represented by the general form: H—first amino acid moiety—second amino acid moiety —OH, wherein the first amino acid moiety comprises an amino acid selected from the group consisting of glycine, asparagine and glutamine; the second amino acid moiety comprises an amino acid selected from the group consisting of lysine, alanine and sarcosine; the first or second amino acid moiety comprises a 6-membered aromatic carbon ring. Preferably, the first amino acid moiety may comprise an amino acid glycine or glutamine and the second amino acid moiety may comprise an amino acid lysine or alanine.

Where the first amino acid moiety comprises an aromatic carbon ring, the amino acid is preferably glutamine. Preferably, the second amino acid moiety comprises the amino acid alanine. Where the second amino acid moiety comprises an aromatic carbon ring, the amino acid is preferably lysine. Preferably, the first amino acid moiety comprises the amino acid glycine. Preferably the aromatic carbon ring is a benzyl or benzoyl group. Preferably the aromatic carbon ring is substituted with at least one of a lower alkyl, alkoxy, halide, nitrile and nitro group, more preferably substituted with at least one of an alkoxy and nitro group. Preferably, the aromatic carbon ring is substituted at the 4-position.

The isopeptides according to the invention have a wide variety of important uses and advantages. Particularly preferred isopeptides for methods, uses and compositions of the invention as described herein are those shown in Table 2.

As discussed, the isopeptide can include a free N-terminal, or a free C-terminal, or both. Isopeptides within the scope of the present invention are often represented herein with free N-terminal and/or C-terminal group. These groups may remain free for some invention uses. However, in another embodiment, the isopeptides can feature blocked C-terminal groups and free N-groups. Alternatively, such isopeptides may have blocked N-groups and free C-terminal groups, or blocked N— and C-terminal groups.

More specific isopeptides within the scope of the invention and having the general Formula II and having the general form H—first amino acid moiety—second amino acid moiety —OH are shown in Table 1. Particularly preferred isopeptides of the invention and having the general Formula II and having the general form H—first amino acid moiety—second amino acid moiety —OH are shown in Table 2.

TABLE 1

| Compound No. | Compound Name |
|---|---|
| 1 | H-Gly-iso-Lys(4-nitrobenzoyl)-OH |
| 2 | H-Gly-iso-Lys(4-fluorobenzoyl)-OH |
| 3 | H-Gly-iso-Lys(4-cyanobenzoyl)-OH |
| 4 | H-Gly-iso-Lys(4-methoxybenzoyl)-OH |
| 5 | H-Gly-iso-Lys(4-chlorobenzoyl)-OH |
| 6 | H-Gly-iso-Lys(benzoyl)-OH |
| 7 | H-Gly-iso-Lys(4-phenoxybenzoyl)-OH |
| 8 | H-Gly-iso-Lys(4-t-butylbenzoyl)-OH |
| 9 | H-Gly-iso-Lys(4-n-butoxybenzoyl)-OH |
| 10 | H-Gly-iso-Lys(4-methylbenzoyl)-OH |
| 11 | H-Gly-iso-Lys(4-ethylbenzoyl)-OH |
| 12 | H-Gly-iso-Lys(4-n-butylbenzoyl)-OH |
| 13 | H-Gly-iso-Lys(4-n-hexylbenzoyl)-OH |
| 14 | H-Gly-iso-Lys(4-n-octylbenzoyl)-OH |
| 15 | H-Gly-iso-Lys(4-pheylbenzoyl)-OH |
| 16 | H-Gly-iso-Lys(4-benzyloxybenzoyl)-OH |
| 17 | H-Gly-iso-Lys(4-ethoxybenzoyl)-OH |
| 18 | H-Gly-D-iso-Lys(4-methoxybenzoyl)-OH |
| 19 | H-Gly-D-iso-Lys(4-nitrobenzoyl)-OH |
| 20 | H-Gly-D-iso-Lys(4-fluorobenzoyl)-OH |
| 21 | H-Gly-D-iso-Lys(4-cyanobenzoyl)-OH |
| 22 | H-Gly-D-iso-Lys(4-chlorobenzoyl)-OH |
| 23 | H-Gly-D-iso-Lys(benzoyl)-OH |
| 24 | H-Ala-iso-Lys(4-nitrobenzoyl)-OH |
| 25 | H-Val-iso-Lys(4-nitrobenzoyl)-OH |
| 26 | H-Ile-iso-Lys(4-nitrobenzoyl)-OH |
| 27 | H-Leu-iso-Lys(4-nitrobenzoyl)-OH |
| 28 | H-Phe-iso-Lys(4-nitrobenzoyl)-OH |
| 29 | H-Trp-iso-Lys(4-nitrobenzoyl)-OH |
| 30 | H-His-iso-Lys(4-nitrobenzoyl)-OH |
| 31 | H-Tyr-iso-Lys(4-nitrobenzoyl)-OH |
| 32 | H-Ala-iso-Lys(4-methoxybenzoyl)-OH |
| 33 | H-Val-iso-Lys(4-methoxybenzoyl)-OH |
| 34 | H-Ile-iso-Lys(4-methoxybenzoyl)-OH |
| 35 | H-Leu-iso-Lys(4-methoxybenzoyl)-OH |
| 36 | H-Phe-iso-Lys(4-methoxybenzoyl)-OH |
| 37 | H-Trp-iso-Lys(4-methoxybenzoyl)-OH |
| 38 | H-His-iso-Lys(4-methoxybenzoyl)-OH |
| 39 | H-Tyr-iso-Lys(4-methoxybenzoyl)-OH |
| 40 | H-Ala-D-iso-Lys(4-nitrobenzoyl)-OH |
| 41 | H-Val-D-iso-Lys(4-nitrobenzoyl)-OH |
| 42 | H-Ile-D-iso-Lys(4-nitrobenzoyl)-OH |
| 43 | H-Leu-D-iso-Lys(4-nitrobenzoyl)-OH |
| 44 | H-Phe-D-iso-Lys(4-nitrobenzoyl)-OH |
| 45 | H-Trp-D-iso-Lys(4-nitrobenzoyl)-OH |
| 46 | H-His-D-iso-Lys(4-nitrobenzoyl)-OH |
| 47 | H-Tyr-D-iso-Lys(4-nitrobenzoyl)-OH |
| 48 | H-Ala-D-iso-Lys(4-methoxybenzoyl)-OH |
| 49 | H-Val-D-iso-Lys(4-methoxybenzoyl)-OH |
| 50 | H-Ile-D-iso-Lys(4-methoxybenzoyl)-OH |
| 51 | H-Leu-D-iso-Lys(4-methoxybenzoyl)-OH |
| 52 | H-Phe-D-iso-Lys(4-methoxybenzoyl)-OH |
| 53 | H-Trp-D-iso-Lys(4-methoxybenzoyl)-OH |
| 54 | H-His-D-iso-Lys(4-methoxybenzoyl)-OH |
| 55 | H-Tyr-D-iso-Lys(4-methoxybenzoyl)-OH |
| 56 | H-iso-Asn(NH(4-trifluoromethylbenzyl))-Ala-OH |
| 57 | H-iso-Asn(NH(4-methoxybenzyl))-Ala-OH |
| 58 | H-iso-Asn(NH(4-nitrobenzyl))-Ala-OH |
| 59 | H-iso-Asn(NH(benzyl))-Ala-OH |
| 60 | H-iso-Asn(NH(4-fluorobenzyl))-Ala-OH |
| 61 | H-iso-Asn(NH(4-chlorobenzyl))-Ala-OH |
| 62 | H-iso-Asn(NH(4-cyanobenzyl))-Ala-OH |
| 63 | H-iso-Asn(NH(4-methylbenzyl))-Ala-OH |
| 64 | H-iso-Asn(NH(4-n-butylbenzyl))-Ala-OH |
| 65 | H-iso-Asn(NH(4-t-butylbenzyl))-Ala-OH |
| 66 | H-iso-Asn(NH(4-n-hexylbenzyl))-Ala-OH |
| 67 | H-iso-Asn(NH(4-n-octylbenzyl))-Ala-OH |
| 68 | H-iso-Asn(NH(4-phenylbenzyl))-Ala-OH |
| 69 | H-iso-Asn(NH(4-phenoxybenzyl))-Ala-OH |
| 70 | H-iso-Asn(NH(4-n-butoxybenzyl))-Ala-OH |
| 71 | H-iso-Asn(NH(4-trifluoromethylbenzyl))-D-Ala-OH |
| 72 | H-iso-Asn(NH(4-methoxybenzyl))-D-Ala-OH |
| 73 | H-iso-Asn(NH(4-nitrobenzyl))-D-Ala-OH |
| 74 | H-iso-Asn(NH(benzyl))-D-Ala-OH |
| 75 | H-iso-Asn(NH(4-fluorobenzyl))-D-Ala-OH |
| 76 | H-iso-Asn(NH(4-chlorobenzyl))-D-Ala-OH |
| 77 | H-iso-Asn(NH(4-cyanobenzyl))-D-Ala-OH |
| 78 | H-iso-Asn(NH(4-methylbenzyl))-D-Ala-OH |
| 79 | H-iso-Asn(NH(4-n-butylbenzyl))-D-Ala-OH |
| 80 | H-iso-Asn(NH(4-t-butylbenzyl))-D-Ala-OH |
| 81 | H-iso-Asn(NH(4-n-hexylbenzyl))-D-Ala-OH |
| 82 | H-iso-Asn(NH(4-n-octylbenzyl))-D-Ala-OH |
| 83 | H-iso-Asn(NH(4-phenylbenzyl))-D-Ala-OH |
| 84 | H-iso-Asn(NH(4-phenoxybenzyl))-D-Ala-OH |
| 85 | H-iso-Asn(NH(4-n-butoxybenzyl))-D-Ala-OH |
| 86 | H-iso-D-Asn(NH(4-trifluoromethylbenzyl))-Ala-OH |
| 87 | H-iso-D-Asn(NH(4-methoxybenzyl))-Ala-OH |
| 88 | H-iso-D-Asn(NH(4-nitrobenzyl))-Ala-OH |
| 89 | H-iso-Asn(NH(4-methoxybenzyl))-Sar-OH |
| 90 | H-iso-Asn(NH(4-methoxybenzyl))-Leu-OH |
| 91 | H-iso-Asn(NH(4-methoxybenzyl))-Phe-OH |
| 92 | H-Gly-iso-Lys(2,4-dinitrobenzoyl)-OH |
| 93 | H-Gly-iso-Lys(2,4-dimethylbenzoyl)-OH |
| 94 | H-Gly-iso-Lys(2,5-dimethylbenzoyl)-OH |
| 95 | H-Gly-iso-Lys(3,5-dimethylbenzoyl)-OH |
| 96 | H-Gly-iso-Lys(2,4-dichlorobenzoyl)-OH |
| 97 | H-Gly-iso-Lys(2,5-dichlorobenzoyl)-OH |
| 98 | H-Gly-iso-Lys(4-fluoro-3-nitrobenzoyl)-OH |
| 99 | H-Gly-iso-Lys(3-fluoro-4-methylbenzoyl)-OH |
| 100 | H-iso-Gln(NH(4-trifluoromethylbenzyl))-Ala-OH |
| 101 | H-iso-Gln(NH(4-methoxybenzyl))-Ala-OH |
| 102 | H-iso-Gln(NH(4-nitrobenzyl))-Ala-OH |
| 103 | H-iso-Gln(NH(benzyl))-Ala-OH |
| 104 | H-iso-Gln(NH(4-fluorobenzyl))-Ala-OH |
| 105 | H-iso-Gln(NH(4-chlorobenzyl))-Ala-OH |
| 106 | H-iso-Gln(NH(4-cyanobenzyl))-Ala-OH |
| 107 | H-iso-Gln(NH(4-methylbenzyl))-Ala-OH |
| 108 | H-iso-Gln(NH(4-n-butylbenzyl))-Ala-OH |
| 109 | H-iso-Gln(NH(4-t-butylbenzyl))-Ala-OH |
| 110 | H-iso-Gln(NH(4-n-hexylbenzyl))-Ala-OH |
| 111 | H-iso-Gln(NH(4-n-octylbenzyl))-Ala-OH |
| 112 | H-iso-Gln(NH(4-phenylbenzyl))-Ala-OH |
| 113 | H-iso-Gln(NH(4-phenoxybenzyl))-Ala-OH |
| 114 | H-iso-Gln(NH(4-n-butoxybenzyl))-Ala-OH |
| 115 | H-iso-Gln(NH(4-trifluoromethylbenzyl))-D-Ala-OH |
| 116 | H-iso-Gln(NH(4-methoxybenzyl))-D-Ala-OH |
| 117 | H-iso-Gln(NH(4-nitrobenzyl))-D-Ala-OH |
| 118 | H-iso-Gln(NH(benzyl))-D-Ala-OH |
| 119 | H-iso-Gln(NH(4-fluorobenzyl))-D-Ala-OH |
| 120 | H-iso-Gln(NH(4-chlorobenzyl))-D-Ala-OH |
| 121 | H-iso-Gln(NH(4-cyanobenzyl))-D-Ala-OH |
| 122 | H-iso-Gln(NH(4-methylbenzyl))-D-Ala-OH |
| 123 | H-iso-Gln(NH(4-n-butylbenzyl))-D-Ala-OH |
| 124 | H-iso-Gln(NH(4-t-butylbenzyl))-D-Ala-OH |
| 125 | H-iso-Gln(NH(4-n-hexylbenzyl))-D-Ala-OH |
| 126 | H-iso-Gln(NH(4-n-octylbenzyl))-D-Ala-OH |
| 127 | H-iso-Gln(NH(4-phenylbenzyl))-D-Ala-OH |
| 128 | H-iso-Gln(NH(4-phenoxybenzyl))-D-Ala-OH |
| 129 | H-iso-Gln(NH(4-n-butoxybenzyl))-D-Ala-OH |
| 130 | H-iso-D-Gln(NH(4-trifluoromethylbenzyl))-Ala-OH |
| 131 | H-iso-D-Gln(NH(4-methoxybenzyl))-Ala-OH |
| 132 | H-iso-D-Gln(NH(4-nitrobenzyl))-Ala-OH |
| 133 | H-iso-Gln(NH(4-methoxybenzyl))-Sar-OH |
| 134 | H-iso-Gln(NH(4-methoxybenzyl))-Leu-OH |
| 135 | H-iso-Gln(NH(4-methoxybenzyl))-Phe-OH |
| 136 | H-Gly-iso-Orn(4-methoxybenzoyl)-OH |
| 137 | H-Gly-iso-Dab(4-methoxybenzoyl)-OH |
| 138 | H-Gly-iso-Dapa(4-methoxybenzoyl)-OH |

More particular isopeptides according to the invention facilitate and/or maintain the intercellular communication mediated by gap junctions. In one aspect, the isopeptides are antiarrhythmic isopeptides which target the same cells targeted by AAP, AAP10, HP5, and/or functional analogs thereof, i.e. the isopeptides are able to modulate the function of these cells by agonizing or antagonizing the function of AAP, PAP10, HP5, and/or functional analogs thereof. The mention of the known AAPs in the present context is to be viewed as one example of gap junction modulating compounds, which are compared to the present isopeptides. The scope of the present invention is, however, not limited to isopeptides having AAP agonistic or antagonistic properties.

The invention also relates to the preparation and use of pharmaceutical compositions for the treatment of pathologies associated with impaired intercellular gap junctional communication and methods for using these compositions.

Further preferred isopeptides in accord with the invention show good activity in one or more of the following assays. Although not necessary to identify orally available isopeptides as represented by Formulae I and II, above, they can be used to further confirm and optionally quantify activity of one or a pool of isopeptides.

Accordingly, and in one embodiment, additionally preferred isopeptides show binding, preferably specific binding, to a tissue, cell, or cell fraction in what is referred to herein as a “standard AAP site binding test”. The test can detect and optionally quantify binding of a subject peptide, e.g., MP, AAP10, HP5, or a functional analog thereof. In one preferred embodiment, the invention isopeptide is a modulator of the function of such a tissue, cell, or cell fraction (i.e., the isopeptide agonizes or antagonizes the function of the antiarrhythmic peptide). In another embodiment, the isopeptide is a modulator of a receptor for the antiarrhythmic peptide (i.e., the isopeptide is an agonist or antagonist of the receptor).

Additionally preferred isopeptides according to Formulae I and II above show good function as a modulator of gap junctional communication (e.g., as agonists or antagonists of AAP). In one aspect, the isopeptides function as an antiarrhythmic drug.

Preferred agonist isopeptides of the invention provide an intracellular conductance (Gj) that is substantially the same as, or is greater than, the Gj of AAP in what is referred to herein as a “standard cardiomyocyte assay”. Preferred antagonist isopeptides provide a Gj that is less (e.g., at least about 10%, or at least about 20% less) than the Gj of AAP and/or block the ability of AAP to normalize the Gj of an ischemic cell, i.e., to return the Gj to substantially the same values found in non-ischemic cells.

Additionally preferred isopeptides according to the invention increase the time to an AV block in a mouse after infusion of CaCl₂, in what is referred to herein as a “standard calcium-induced arrhythmia assay.” Preferably, the isopeptides provide at least about 50% of the activity of AAP, preferably at least about 70% of the activity of AAP, more preferably substantially the same activity of AAP (i.e., show time lags of approximately the same duration).

Isopeptides of the invention may additionally show decreases in the incidence of reentry arrhythmias or in the size of an infarct zone observed in what is referred to herein as a “standard ventricular reentry assay.” Preferably, the isopeptides provide at least about 50% of the activity of AAP, preferably at least about 70% of the activity of AAP, more preferably substantially the same activity of AAP in this assay (i.e., providing similar decreases in incidence or infarct zones of similar or smaller size).

There have been attempts to improve the oral availability of certain compounds by enhancing contact with an intestinal peptide transporter protein called PepT1. Much is known about the PepT1 transporter system. See Bailey, P. D., et al. (2000) Angew. Chem. Int. Ed. 39: 506; and references cited therein. In one aspect of the invention the potential oral availability of the present isopeptides may be examined. One empirical assay to validate the predictive Bailey assay described above, is a “standard in vivo oral availability assay”. In this assay, an isopeptide is orally administered to a mammal and blood samples are taken over time. The concentration of the isopeptide is determined at different time intervals using standard protein quantitation assays such as LC/MS/MS to calculate an area under the curve (AUC) from a plot of plasma protein concentration vs. time, using routine methods known in the art. Preferably, different doses of isopeptides are administered to a plurality of animals in parallel to identify those isopeptides that show a dose-proportional increase in maximum plasma concentration and AUC values. As a control, the same concentrations of isopeptide may be administered intravenously and the area under AUC obtained for oral administration can be compared to the AUC obtained for intravenous administration. See, e.g., Milo Gibal (1991) Biopharmaceutics and Pharmacology, 4^{th} edition (Lea and Sediger). Isopeptides with good oral availability are those which are observed in plasma within less than about 30 minutes after administration.

Isopeptides which show good oral availability according to the standard in vivo oral availability assay described above, may or may not be in substantial agreement with the Bailey assay. However for those isopeptides that do not show such agreement (and also for those that do), the isopeptides will be observed in the plasma within less than about 30 minutes after oral administration as determined by the standard in vivo oral availability assay.

An hPepT1 binding assay may be performed as an initial screen for isopeptides which are then screened for in a standard in vivo oral availability assay and/or may be performed to confirm the results of a standard in vivo oral availability assay; however, the standard in vivo oral availability assay provides the most meaningful test of the isopeptide oral availability.

Additionally preferred isopeptides as represented by Formula I and II above, exhibit a good half-life according to what is referred to herein as an “in vitro plasma stability assay” or related phrase. Such isopeptides may be in substantial agreement with the foregoing PepT1 substrate template model (Bailey assay). However, for some isopeptides there may be little or no agreement with the model. Isopeptides that show a good stability in the assay have in one embodiment a half-life of more than about 48 hours, such as more than 24 hours, for example more than 12 hours, such as more than 6 hours, for example more than 3 hours, such as more than 1 hour, for example more than 30 minutes, such as more than 20 minutes, for example more than 15 minutes, such as more than 10 minutes, for example more than 5 minutes, such as more than 1 minute. In this embodiment, the isopeptides of the invention may show enhanced stability in the bloodstream.

Osteoporosis

There is understanding that GJIC is important in bone formation. Additionally preferred isopeptides additionally, or alternatively, increase osteoblast activity in what is referred to herein as a “standard osteoblast activity assay” which measures either calcium wave formation and/or alkaline phosphatase activity of osteoblast cells in the presence of isopeptides. Preferably, such isopeptides increased calcium wave activity, manifested as an increase in the number of cells involved in a wave (as determined by measuring levels of intracellular Ca²⁺ using a calcium sensitive fluorescent dye, such as fura-2 and counting the number of cells which fluoresce). Alkaline phosphatase activity also can be used to provide a measure of osteoblast activity using standard colorimetric assays. Agonist isopeptides according to the invention provide at least about 10% of the activity of AAP in such an assay, such as at least about 20% activity, for example at least about 30% activity, such as at least about 40% activity, for example at least about 50% of the activity of AAP, preferably, at least about 70% activity, and still more preferably, 100% or greater activity of the activity of AAP.

Cancer

Preferred isopeptides according to the invention, alternatively, or additionally, decrease GJIC inhibition mediated by tumor promoters such as DTT, in what is referred to herein as a “standard tumor promoter assay.” Preferably, the isopeptides show decreases in GJIC inhibition, which are at least 50%, preferably 70%, and more preferably 100% or greater, than decreases observed for AAP.

As discussed, it is an object of the invention to provide isopeptides that modulate gap junction intercellular communication (GJIC). Thus, many isopeptides in accord with the invention may include one or more of the following features: the ability to decrease cellular uncoupling, to normalize dispersion of action potential duration, and to normalize conduction velocity, the ability to control of the cellular quantity of gap junctions normalizing (up-regulating or down-regulating as needed) the expression of connexins; to normalize degradation of gap junctions (inhibit or enhance), to normalize cellular trafficking of connexins to the plasma membrane (increase or decrease); to facilitate assembly of connexins into functional gap junctions; to normalize opening of existing gap junctions, e.g., inducing or enhancing opening when they have been closed or gated by inhibitors (e.g., such as by mediating or enhancing hyperphosphorylation of the cytoplasmic carboxy terminal domain of one or more connexins (e.g., such as Cx43)) or closing these when they are aberrantly opened (e.g., as in Charcot-Marie-Tooth disease).

Preferred isopeptides according to the invention, alternatively, or additionally, decrease GJIC inhibition mediated by tumor promoters such as DTT, in what is referred to herein as a “standard tumor promoter assay.” Preferably, the isopeptides show decreases in GJIC inhibition which are at least 50%, preferably 70%, and more prefereably 100% or greater, than decreases observed for AAP.

Particular assays useful for identifying and optionally quantifying the activity of preferred invention isopeptides are described below.

A. Standard Plasma Stability Assays

The invention also provides isopeptides that have enhanced stability in vitro or in vivo. In one aspect the peptide comprises a peptide bond that is alkylated or otherwise modified to stabilize the peptide against enzymatic degradation. In another aspect, the peptide comprises one or more D-amino acids. In a further aspect, the peptide has enhanced stability in a standard stability assay.

In one aspect, an in vitro plasma stability assay is performed as described in PCT/US02/05773, filed Feb. 22, 2002.

As disclosed in the PCT/US02/05773 application, peptides can be incubated in plasma or serum and samples taken at regular intervals for analysis by HPLC or LC/MS/MS, to quantitate the amount of undegraded peptide. Appropriate conditions (column, solvent, gradient, and temperature) for such analyses are estimated to ensure that the peptide peak and the plasma peaks do not have the same retention time. This is done by subsequent injections of a peptide, plasma, and a co-injection with the peptide and the plasma, followed by optimization of LC method parameters until a satisfactory separation is obtained. A control plasma sample without the peptide, treated in the same manner, also can be taken and evaluated. The samples may include, but are not limited to, a blank, the peptide at a suitable concentration (e.g., 0.1 mg/mL), plasma without peptide, one or more samples for t=0, and one or more samples at each regular interval. Preferably, multiple samples are taken in parallel. The sample concentrations (peak height in mAU or ion counts) can be plotted vs. time and fitted to a function describing a mono exponential decay (e.g., using a standard Excel package). Preferably, a peptide according to the invention has a half-life of more than about 48 hours, such as more than 24 hours, for example more than 12 hours, such as more than 6 hours, for example more than 3 hours, such as more than 1 hour, for example more than 30 minutes as determined using this assay.

Plasma stability can be examined in vivo using standard assays. For example, isopeptides may be administered to a mammal, such as a rat, by bolus injections in volumes of about 1 ml/kg for both i.v. and p.o. dosing. Preferably, isopeptides are tested in parallel with control samples such as buffer or an antiarrythmic peptide with a known stability. Blood samples are collected at different time periods (e.g., at B.D. 5, 15, 30, 60, 90, 120, 180, and 240 minutes, where B.D. refers to before dose). Amounts of isopeptides in samples can be quantitated using methods routine in the art, such as LC/MS/MS. For example, the concentrations of isopeptides in plasma samples may be calculated from an external standard curve covering ranges of peptide from 1.00 to 1000 nM. The plasma concentrations versus time data can be used for pharmacokinetic modeling in WinNonLin 3.5 (Pharsight, Mountain view, Calif.) using non-compartmental analysis and the resulting parameters of AUC, Fpo, Clb, t1/2, Cmax and tmax can be determined as is known in the art.

B. Standard Oral Availability Assay

It is a preferred aspect of the invention to provide isopeptides with enhanced availability in vivo. Absorption of isopeptides after oral administration is often limited because they are degraded by either enzymes in the gastrointestinal (GI) tract or by enzymes in the intracellular lumen of the enterocytes. Further, the physico-chemical properties of isopeptides, especially their large hydrogen-bonding potential, makes it difficult for these molecules to permeate the enterocytes by transcellular passive diffusion.

Preferred isopeptides according to the invention are therefore isopeptides that have affinity for an hPepT1 transporter or an analog thereof. The three-dimensional conformation and key binding sites of peptide compounds which bind the PepT1 transporter are described by Bailey, P. D., et al., 2000, supra, and desired peptides can be modeled in silico to optimally fit within this binding site, as described above (see, e.g., Bailey, P. D., et al., (2000), supra; Vinter, J. G., (1996) J. Comput Aided Des. 10: 417).

The oral availability of a peptide comprising a structure as shown in Formula I, Formula II, Table 1, or more generally identified using the Bailey assay, may be evaluated for its ability to bind to a PepT1 transporter, preferably an hPepT1 transporter. For example, a PepT1 cDNA, preferably, an hPepT1 cDNA (see, e.g., Covitz, K. M., et al., (1996) Pharm. Res. 13(11): 1631-34) may be expressed in a Xenopus oocyte expression system and the uptake of labeled peptide into the oocyte can be monitored to approximate Ki values as described in Temple, C. S., et al. (1996) J. Physiol. (London) 494: 795; Meredith, D., et al., (1998) J. Physiol. (London) 512: 629.

In one aspect of the invention, a standard in vivo oral availability assay is performed to determine the oral availability of an isopeptide which has been modeled to optimally conform to the Bailey substrate template, as discussed above. In this assay, an isopeptide is orally administered to a mammal, such as a rat, in anorally administrable form (e.g., as part of a food pellet or in water), while at the same time, the same concentration of peptide is administered i.v. (e.g., through a catheter inserted into the femoral vein and artery). Isopeptides may be administered as bolus injections at concentrations ranging from 10⁻⁵-10⁻¹⁰ in volumes of 1 ml/kg for both oral and i.v. dosing. The animals are given 500 I.U. of heparin i.v. 5 minutes before the first blood sample is taken. A control blood sample, “before dose”, or B.D. sample, is collected approximately 5 minutes before administration of the isopeptides. A sample of the dosing solution (e.g., 100 μl of water comprising 10⁻⁵-10⁻¹⁰ M of peptide) is retained for concentration determination. Blood samples are collected at t=B.D. 5,15, 30, 60, 90, 120, 180, and 240 minutes.

Blood is collected in labeled ice-chilled EDTA stabilized blood sample vials and stored on ice until quickly centrifuged at 4° C. for 5 minutes (10,000×g). Plasma (100 μl) is harvested, transferred to a labeled polypropylene microcentrifuge vial (e.g., 0.5 ml eppendorf), frozen on ice and stored at −20° C. until further analysis. Approximately 40 μl of the filtrate is injected onto an HPLC column (XterraMS C18, 3×50 mm, 3.5 μm particles) and eluted using a linear gradient from 0 to 100% B in 4.0 minutes. The column is washed for 2.9 minutes in buffer B (0.1% formic acid in acetonitrile or another suitable buffer) and equilibrated for 5 minutes in Buffer A (0.1% formic acid in water or another suitable buffer) prior to the next injection of sample. Mass spectrometry is performed using methods routine in the art and as described further below in Examples 1 and 2.

The concentrations of compounds in the plasma samples are calculated from an external standard curve covering the range from 1.00 to 1000 nM. The plasma concentrations versus time data are used for pharmacokinetic modeling in WinNonLin 3.5 (Pharsight, Mountain view, Calif.) using non-compartmental analysis and AUC values are determined as is known in the art. Preferably, orally available isopeptides according to the invention are observed in significant levels in plasma within about 30 minutes or less. AUC values observed in animals receiving i.v. administrations of peptide are used to evaluate such effects as clearance and half-life which should be the same in the two systems.

C. Standard Cardiomyocyte Assays

In one aspect, an isopeptide according to the invention is administered to a cardiac cell and gap junction function is evaluated. Optimal isopeptides for such procedures can be identified in standard cardiomycte assays. In one aspect, cardiac cells are isolated from a mammal, such as a guinea pig hearts by perfusion with collagenase according to the Langendorf method. The cells are exposed to isopeptide and evaluated for GJIC by patch clamp using methods known in the art. Intercellular conductance (Gj) using the formula:

$${G}_{j} = \frac{Δ {I}_{p}}{Δ {U}_{j}} = \frac{{I}_{p , pulse} - {I}_{p , rest}}{{U}_{p} - {U}_{a}} \qquad ( Equation 1 )$$

Where Ip,pulse and Ip,rest represent the current in the passive cell during the pulse and before the pulse respectively, and Up and Ua represent the voltage of the passive and active cell. The change in Gj value upon isopeptide administration is analyzed by comparing the relative changes in Gj. For example, the relative Gj as a function of time before, and during, stimulation with isopeptide (e.g., at about 10⁻⁸ M) can be determined. Preferably, the isopeptide provides a Gj, which is substantially the same as the Gj (±10%) of an antiarrhythmic peptide such as AAP, AAP10, HP5, and functional analogs thereof. In one aspect, the cell is an ischemic cell, and the isopeptide provides a Gj, which is substantially the same as that of a non-ischemic cell (±20%, preferably, ±10%).

Additional details concerning performing cardiomyocyte assays are provided in PCT/US02105773, filed Feb. 22, 2002.

D. Standard Calcium-Induced Arrhythmia Assay

Isopeptides suitable for administration to cardiac cells can be identified in an in vivo model of calcium-induced arrhythmias according to the model of Lynch et al. (1981) J Cardiovasc. Pharmacol. 3: 49-60. Mice (25-30 g) are anaesthetized with a neurolept anaesthetic combination and an i.v. cannula is inserted into the tail vein. A lead II ECG signal is recorded continuously by positioning a stainless steel ECG electrodes on the right forelimb and on the left hind limb. The ground electrode is placed on the right hind limb. The signal is amplified (x 5.000-10.000) and filtered (0.1-150 Hz) via a Hugo Sachs Electronic model 689 ECG module. The analog signal is digitized via a 12 bit data acquisition board (Data Translation model DT321) and sampled at 1000 Hz using the Notocord HEM 3.1 software for Windows NT. After a 10-minute equilibration period, the test sample of peptide is injected into the tail vein. Mice pre-treated with buffer are tested as a measure of the control level in untreated animals. The injection volume is 100 μl in all experiments.

Infusion of CaCl₂ (30 mg/ml, 0.1 ml/min≈100 mg/kg/min (calcium chloride-2-hydrate, Riedel-de Haën, Germany)) is started 3 min after i.v. administration of drug or vehicle. The time lag to onset of 2^{nd} degree AV-block is determined as the time from the start of CaCl2 infusion until the first arrhythmic event occurs. An event of 2^{nd} degree AV-block is defined as intermittent failure of the AV conduction characterised by a P-wave without the concomitant QRS complex.

Responses are expressed relative to the time until 2^{nd} degree AV-block occurred in vehicle treated mice. The maximal effect of compounds (e.g., isopeptides, AAP, AAP10 or controls) is determined. Preferably, isopeptides according to the invention have antiarrhythmic activity comparable to the compounds AAP, AAP10, HP5, or a functional analog thereof, i.e., the peptides increase the time to an AV block in a mouse after infusion of CaCl₂. Preferably, the isopeptides provide at least about 40% of the activity of MP, for example at least about 50% of the activity of MP, such as about 60% of the activity of MP, for example at least about 70% of the activity of AAP, such as at least about 80% of the activity of AAP, for example at least about 90% of the activity of AAP, for example at least about substantially the same activity of AAP, such as about 110% of the activity of AAP, for example at least about 120% of the activity of AAP, such as at least about 130% of the activity of AAP, for example at least about 140% of the activity of MP, such as about 150% of the activity of AAP, for example at least about 160% of the activity of AAP, such as at least about 170% of the activity of AAP, for example at least about 180% of the activity of AAP, preferably at least about 190% of the activity of AAP, more preferably at least about 200 or greater % of the activity of MP (i.e., the peptides show time lags of approximately the same duration as induced by AAP).

E. Standard Osteoblast Activity Assay

Modulation of intercellular communication represents a mechanism by which osteotropic factors regulate the activity of bone forming cells. Therefore, in one aspect, isopeptides according to the invention are used to increase osteoblast activity by increasing gap junctional communication between bone cells, thereby enhancing bone formation in vivo.

The efficacy of an isopeptide according to the invention may be assayed in preliminarily in human osteblast cells (hOB), for example by measuring calcium wave activity and/or alkaline phosphatase activity.

In one aspect, cells are isolated from human bone marrow obtained by puncture of the posterior iliac spine of healthy volunteers (aged 20-36): 10-15 ml marrow material was collected in 15 ml PBS+Ca,Mg (Life Technologies, Cat. No.14040) with 100 U/ml Heparin (Sigma, Cat. No. H-3149). The mononuclear fraction of the marrow is isolated on a Lymphoprep gradient (Nycomed Pharma, Cat.No. 1001967), by centrifugation at 2200 rpm for 30 min. After harvesting, the mononuclear fraction is washed once with culture medium and centrifuged at 1800 rpm for 10 min. Subsequently cells are counted and plated in culture medium at 5×10⁶ cells/100 mm dish. hOB medium (all reagents obtained from Life Technologies): MEM w/o Phenol Red W/Glutamax (Cat.No. 041-93013) supplemented with 10% heat inactivated fetal calf serum (Cat.No. 10106) and 0.1% Penicillin/Streptomycin (Cat.No. 15140). Medium is changed the following day and the cells are cultured at 37° C. in 5% CO₂ with medium change every 7 days. After 4-6 weeks of culture, the cells will reach 70% confluence. The medium is then supplemented with 100 nM Dexamethasone (Sigma, Cat.No. D-4902) for 7 days. Cells are then plated for video imaging experiments: a 25 mm #1 glass coverslip is placed in a 35 mm dish (or each well of a 6-well multidish), cells are plated at 2.5×10⁵ cells/coversip and cultured for 2-3 days before use. ROS 17/2.8 cells are cultured in 100 mm dishes at 37° C. with 5% CO₂ and medium changed every 2-3 days. ROS medium (all reagents obtained from Life Technologies): MEM (Cat.No. 31095) is supplemented with 10% heat-inactivated calf serum (Cat.No. 16170), 1% NEM (Cat.No. 11140), 1% Sodium Pyruvate (Cat.No. 11360), 1% L-Glutamine (Cat.No. 25030) and 0.1% Penicillin/Streptomycin (Cat.No. 15140). For video imaging experiments, cells are plated on coverslips at 2-3×10⁵ cells/coverslip and cultured for 2-3 days before use.

The cells cultured on coverslips are loaded with 5 μM fura-2-AM (Molecular Probes, Cat.No. F-1221), for 30 minutes at 37° C., and incubated in fresh medium for 20 minutes. Coverslips are then affixed to a PDMI-2 culture chamber (Medical Systems Corp.), maintained at 37° C. with superfused CO₂, on a Zeiss Axiovert microscope. Intercellular calcium waves are induced by mechanical stimulation of a single cell using a borosilicate glass micro pipette affixed to an Eppendorf 5171 micromanipulator.

Imaging is performed using a MetaMorph imaging system (Universal Imaging). The excitation light (340 and 380 nm) is provided by a monochromator (T.I.L.L. Photonics GmbH). Images are acquired with an intensified CCD camera (Dage MTI) and digitized with a Matrox MVP image processing board. The number of cells involved in a calcium wave in the presence and absence of peptide can be used to provide a measure of increase in GJIC.

In one aspect, administration of an isopeptide increases the number of cells involved in a wave at least about two-fold compared to cells which have been exposed to a control, such as buffer. In another aspect, administration of an isopeptide decreases the number of cells involved in a wave by at least about two-fold. Agonist isopeptides according to the invention provide at least about 10% of the activity of AAP in such an assay, such as at least about 20% activity, for example at least about 30% activity, such as at least about 40% activity, for example at least about 50% of the activity of AAP, preferably, at least about 70% activity, and still more preferably, 100% or greater activity of the activity of MP.

Cells also can be measured for the presence of alkaline phosphatase activity to provide a general measure of osteoblast activity. In one aspect, cells are plated in 96-well plates at a concentration of 8000 cells/well (hOB) or 3000 cells/well (ROS) in 200 μl normal culture medium. On day 4 (or day 3 for ROS cells), cells are washed with 200 μl MEM, 0.1% BSA (Sigma, Cat.No. A-9418). Samples comprising a suitable medium (e.g., 200 μl MEM, 0.1% BSA) containing various concentrations of peptides, control, AAP or AAP10 are added to the cells, and culture is continued for about 4 days (2 days for ROS cells).

On about day 8 (preferably day 5 for ROS cells), cells are assayed for alkaline phosphatase using an Alkaline Phosphatase (ALP) assay such as is known in the art. ALP assays are generally colorimetric endpoint methods for measuring enzyme activity, and can be performed using an Alkaline Phosphatase Kit (Sigma, Cat.No. 104-LL). Preferably, cells are washed once with 200 μl PBS+Ca, Mg, 100 μl Alkaline Buffer Solution is added to each well and the cells are incubated at 37° C. for 10 minutes. 100 μl Substrate Solution is subsequently added to each well and the plate is incubated at 37° C. for 30 min. 100 μl 2.0 N NaOH is added to each well to stop the reaction. Absorbance is measured using a plate reader at 405 nm.

Agonist isopeptides according to the invention provide at least about a 5% increase in alkaline phosphatase production relative to isotonic saline, preferably, at least about 10% increase in alkaline phosphatase production relative to isotonic saline, and still more preferably a 15% or greater increase in alkaline phosphatase production relative to isotonic saline. The increase in production of alkaline phosphatase is a measure for increased activity of osteoblasts and accordingly a measure for an increase in bone formation.

F. Standard Tumor Promotor Assay

The compound 1,1-bis(p-chlorophenyl)-2,2,2-trichlorethane, also known as the insecticide DDT, is an inhibitor of gap junctional communication, and has tumor promoting abilities. It inhibits cell-to-cell communication by reducing the gap junction number and size, and exposure to DDT is associated with decreased cellular levels of phosphorylated (active) forms of the gap junction protein Cx43. These actions are considered pivotal for the compound's oncogenic properties (X. Guan, et al. (1996) Carcinogenesis, 17 1791-1798; R. J. Ruch, et al. (1994) Carcinogenesis, 15 301-306); B. V. Madhukar, et al. (1996) Cancer Lett. 106 117-123). As a means of monitoring the therapeutic efficacy of the isopeptides, the effects of the isopeptides on DDT-induced uncoupling in human osteoblast cells can be determined. Thus, in one aspect, isopeptides according to the invention are used to inhibit or prevent tumor-promoter induced decreases of GJIC (W. K. Hong, et al. (1997) Science, 278 1073-1077).

In one exemplary assay, human osteoblast cells are isolated from human bone marrow obtained by puncture of the posterior iliac spine of healthy volunteers (aged 20-36). Approximately 10-15 ml of bone marrow material are collected in 15 ml PBS+Ca, Mg (Life Technologies, Cat.No. 14040) with 100 U/ml Heparin (Sigma, Cat.No. H-3149). The mononuclear fraction of the marrow is isolated on a Lymphoprep gradient (Nycomed Pharma, Cat.No. 1001967), by centrifugation at 2200 rpm for 30 minutes.

After harvesting, the mononuclear fraction is washed once with culture medium and centrifuged at 1800 rpm for 10 minutes. Subsequently cells are counted and plated in culture medium at 8×10⁶ cells/100 mm dish. Medium is changed the following day and the cells are cultured at 37° C. in 5% CO₂ with medium changes every 7 days. After 3-4 weeks of culture, the cells typically reach 70% confluence. The medium is then supplemented with 100 nM Dexamethasone (Sigma, Cat.No. D-4902) for 7 days. Cells are then plated for video imaging experiments. Generally, cells are plated at 2.5×10⁵ cells/coverslip and are cultured for 2-3 days before imaging.

After culturing, cells are affixed to a PDMI-2 culture chamber (Medical Systems Corp.), maintained at 37° C. with superfused CO₂, on a Zeiss Axiovert microscope. Microinjections are performed using a micropipette is loaded with a 10 mM Lucifer Yellow solution (Sigma, Cat.No. L-0259). A cell in the monolayer is carefully injected with LY for 30 seconds; the micropipette is removed from the cell and after 30 seconds the number of cells that show dye transfer is counted. For a subset of cell cultures, DDT is added to the medium in a final concentration of 13 μM, and is left on for 60 minutes. Images of cells are acquired with an intensified CCD camera (Dage MTI) and digitized with a Matrox MVP image processing board, using the MetaMorph imaging software (Universal Imaging).

Under control conditions (no DDT treatment), the dye generally spreads to a median of 14.5 cells (n=12). DDT-exposed cells typically show decreased cellular coupling with a median of 7 (n=13).

Isopeptides are added to the bathing solution in a final concentration of 10⁻⁸ mol/l, and after 10 minutes, another microinjection is performed. Preferably, agonist isopeptides according to the invention show an increase in cell-to-cell dye transfer. More preferably, this increase is significantly different from control samples (without peptides) as determined using routine statistical tests, such as the Wilcoxon non-parametric statistical test (with p<0.05). Preferably, the isopeptides show decreases in GJIC inhibition which are at least about 50%, preferably about 70%, and more preferably, about 100% or greater, than decreases observed for AAP. This assay can be used to identify candidate isopeptides with the highest therapeutic efficacy in reversing the decreased intercellular coupling related to tumor promotion and in one aspect, such isopeptides are administered to individuals at risk for developing or having cancer. An isopeptide may be used alone or in combination with other isopeptides and/or in a combination therapy with other anti-cancer agents.

Still other assays may be performed to identify isopeptides which elicit substantially the same physiological responses as the antiarrhythmic peptides AAP, AAP10, HP, and their functional analogs (e.g., to identify agonists) or which inhibit or suppress these physiological responses (e.g., to identify antagonists). Suitable assays include, but are not limited to: assays to measure cAMP formation in cells (e.g., CHO cells); cAMP efficacy assays (e.g., measuring inhibition of forskoline-stimulated cAMP formation of APP-like compounds in CHO cells); phosphoinositol turnover in cardiomyocytes (Meier et al.) (E. Meier, et al. (1997) Drug Development Research, 40: 1-16); and responses to glucose and oxygen deprivation.

A number of standard assays are detailed above. Additional assays are described in PCT/US02/05773, filed Feb. 22, 2002, the entirety of which is incorporated herein by reference. These assays are exemplary only and other suitable assays that may be developed and become standardized are encompassed within the scope of the invention.

Pharmaceutical Compositions

The invention also concerns a pharmaceutical composition comprising one or more of any of the isopeptides described above, in combination with a pharmaceutically acceptable carrier and/or diluent.

Formulation/Carrier

For therapeutic use, the chosen isopeptide of the invention is formulated with a carrier that is pharmaceutically acceptable and is appropriate for delivering the isopeptide by the chosen route of administration. For the purpose of the present invention, peripheral parenteral routes include intravenous, intra muscular, subcutaneous, and intra peritoneal routes of administration. Certain compounds used in the present invention may also be ameable to administration by the oral, rectal, nasal, or lower respiratory routes. These are so-called non-parenteral routes. The present pharmaceutical composition comprises an isopeptide of the invention, or a salt thereof and a pharmaceutically acceptable carrier. Suitable pharmaceutically acceptable carriers are those used conventionally with peptide-based drugs, such as diluents, excipients and the like. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Ph buffering agents may be histidine, or sodium acetate. Preservatives, stabilizers, dyes and even flavoring agents may be provided in the pharmaceutical composition. For example, phenol sodium benzoate, sorbic acid and esters of p-hydroxybenzoic acid may be added as preservatives. In addition, antioxidants and suspending agents may be used, e.g. SDS, ascorbic acid, methionine, carboxy methyl cellulose, EDTA, polyethylene glycol, and Troeen.

The pharmaceutical compositions of the present invention may be formulated and used as tablets, capsules or elixers for oral administration; suppositories for rectal administration; sterile solutions and suspensions for injectable administration; and the like. The dose and method of administration can be tailored to achieve optimal efficacy but will depend on such factors as weight, diet, concurrent medication and other factors, which those skilled in the medical arts will recognize.

The pharmaceutical carrier or diluent employed may be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water.

Similarly, the carrier or diluent may include any sustained release material known in the art, such as glyceryl monostearate or glyceryl distearate, alone or mixed with a wax.

Parenteral Administration

When administration is to be parenteral, such as subcutanous on a daily basis, injectable pharmaceutical compositions can be prepared in conventional forms, either as aqueous solutions or suspensions lyophilized, solid forms suitable for reconstitution immediately before me or suspension in liquid prior to injection, or as emulsions. Suitable excipients are, for example, water, saline, dextrose, mannitol, lactose, lecithin, albumin, sodium glutamate, and cysteine hydrochloride. In addition, if desired, the injectable pharmaceutical compositions may contain minor amounts of nontoxic auxilliary substances, such as wetting agents, or pH buffering agents. Absorption enhancing preparations (e.g., liposomes) may be utilized. In one embodiment of the invention, the compounds are formulated for administration by infusion, e.g., when used as liquid nutritional supplements for individuals on total parenteral nutrition therapy (for example neonatals), or by injection, for example sub-cutaneously, intraperitoneal or intravenously, and are accordingly utilized as aqueous solutions in sterile and pyrogen-free form and optionally buffered to physiologically tolerable pH, e.g., a slightly acidic or physiological pH. Formulation for intramuscular administration may be based on solutions or suspensions in plant oil, e.g. canola oil, corn oil or soy bean oil. These oil based formulations may be stabilized by antioxidants e.g. BHA (Butylated Hydroxianisole) and BHT (Butylated Hydroxytoluene).

Thus, the present isopeptide compounds may be administered in a vehicle, such as distilled water or in saline, phosphate buffered saline, 5% dextrose solutions or oils. The solubility of the present isopeptides may be enhanced, if desired, by incorporating a solubility enhancer, such as detergents and emulsifiers.

The aqueous carrier or vehicle can be supplemented for use as injectables with an amount of gelatin that serves to depot the isopeptide at or near the site of injection, for its slow release to the desired site of action. Alternative gelling agents, such as hyaluronic acid, may also be useful as depoting agents.

The isopeptides of the invention may also be formulated as a slow release implantation device for extended and sustained administration of the isopeptides. Such sustained release formulations may be in the form of a patch positioned externally on the body. Examples of sustained release formulations include composites of biocompatible polymers, such as poly(lactic acid), poly(lactic-co-glycolic acid), methylcellulose, hyaluronic acid, collagen, liposomes and the like. Sustained release formulations are of particular interest when it is desirable to provide a high local concentration of an isopeptide of the invention.

The isopeptide may be utilized in the form of a sterile-filled vial or ampoule containing an intestinotrophic amount of the peptide, in either unit dose or multi-dose amounts. The vial or ampoule may contain the isopeptide and the desired carrier, as an administration-ready formulation. Alternatively, the vial or ampoule may contain the isopeptide in a form, such as a lyophilized form, suitable for reconstitution in a suitable carrier, such as sterile water or phosphate-buffered saline.

Non-Parenteral Administration

As an alternative to injectable formulations, the isopeptide may be formulated for administration by other routes. Oral dosage forms, such as tablets, capsules and the like, can be formulated in accordance with standard pharmaceutical practice.

It will be appreciated that the actual preferred amounts of active compounds used in a given therapy will vary according to e.g. the specific compound being utilized, the particular composition formulated, the mode of administration and characteristics of the subject, e.g. the species, sex, weight, general health and age of the subject. Optimal administration rates for a given protocol of administration may be readily ascertained by those skilled in the art using conventional dosage determination tests conducted with regard to the foregoing guidelines. Suitable dose ranges may include from about 1 mg/kg to about 100 mg/kg of body weight per day.

Treatment Methods

In one aspect, the invention provides a method of administering to an individual having, or at risk of developing, a condition associated with impaired GJIC, a therapeutically effective amount of any of the isopeptides described above. Individuals who may be treated using isopeptides according to the invention include, but are not limited to, animals, preferably mammals, e.g., rodents (including mice, rats, hamsters, and lagomorphs, such as rabbits), dogs, pigs, goats (generally any domestic animal), and primates. In one preferred aspect, an individual is a human being.

Examples of conditions which can be treated include, but are not limited to, cardiovascular disease, inflammation of airway epithelium, disorders of alveolar tissue, bladder incontinence, impaired hearing due to diseases of the cochlea, endothelial lesions, diabetic retinopathy and diabetic neuropathy, ischemia of the central nervous system and spinal cord, dental tissue disorders including periodontal disease, kidney diseases, failures of bone marrow transplantation, wounds, erectile dysfunction, urinary bladder incontinence, neuropathic pain, subchronic and chronic inflammation, cancer and failures of bone marrow and stem cell transplantation, conditions which arise during transplantation of cells and tissues or during medical procedures such as surgery; as well as conditions caused by an excess of reactive oxygen species and/or free radicals and/or nitric oxide.

In one preferred aspect, the invention provides a pharmacologically active antiarrhythmic isopeptide, and the use thereof, for treatment of arrhythmias and thrombotic complications arising during cardiovascular disorders, such as acute ischemic heart disease (e.g., stable angina pectoris, unstable angina pectoris, acute myocardial infarction), congestive heart failure (e.g., systolic, diastolic, high-output, low-output, right or left sided heart failure), congenital heart diseases, corpulmonale, cardiomyopathies, myocarditis, hypertensive heart disease, during coronary revascularization, and the like.

In specific embodiments, an antiarrhythmic isopeptide according to the present invention is used to treat and/or prevent bradyarrhythmias (e.g., due to disease in sinus node, AV node, bundle of His, right or left bundle branch), and tachyarrhythmias associated with reentry (e.g., atrial premature complexes, AV junctional complexes, ventricular premature complexes, atrial fibrillation, atrial flutter, paroxymal supraventricular tachycardia, sinus node reentrant tachycardia, AV nodal reentrant tachycardia, and non-sustained ventricular tachycardia) either alone or in combination with other antiarrhythmic compounds, such as class I agents (e.g., lidocaine), class II agents (e.g., metoprolol or propranolol), class III agents (e.g., amiodarone or sotalol) or class IV agents (e.g., verapamil).

Additionally, or alternatively, isopeptides according to the invention are used to treat one or more of: a reentry arrhythmia; ventricular reentry (e.g., such as arises during acute myocardial infarction, chronic myocardial infarction, stable angina pectoris and unstable angina pectoris); infectious or autonomic cardiomyopathy; atrial fibrillation;repolarization alternans; monomorphic ventricular tachycardia; T-wave alternans; bradyarrhythmias; and generally, reduced contractility of cardiac tissue, thrombosis and the like.

Osteoporosis

In a further aspect, isopeptides according to the invention are used to prevent and/or treatment of osteoporosis or other pathologies affecting bone formation, growth or maintenance. Isopeptides which are able to normalize the attenuated GJIC between human osteoblast during hypoxia are particularly suitable for the treatment of bone diseases with impaired bone formation relative to bone resorption. Optimal isopeptides for use in such methods can be selected in assays for increased alkaline phosphatase (ALP) activity in osteoblasts, which provides a means to monitor cell viability and growth as a consequence of proper maintenance of GJIC. In one aspect, human osteoblasts are stimulated with different concentrations of isopeptides from 1×10⁻¹³ to 1×10⁻⁶ mol/l, and compared to untreated controls. Under normal culture conditions, isopeptides preferably increase ALP activity. Even more preferably, the isopeptides stimulate ALP activity during hypoxic conditions at concentrations ranging from 10⁻¹¹ to 10⁻⁸ mol/l. The assay can thus be used to optimize isopeptide compositions for the treatment and/or prevention of bone diseases associated with poor vascularization, hypoxia and ischemia in bone tissue.

Joint Diseases

In another aspect, isopeptides according to the invention are used for the prevention and/or treatment of joint diseases that involves impaired cell-to-cell coupling. For example, the isopeptides can be used for the prevention and/or treatment of joint diseases that involve metabolic stress. These would include any form of arthritis associated with decreased vascularization or healing of fractured cartilage tissue.

Cancer

In still another aspect, isopeptides according to the invention are used to treat cancer. Carcinogenesis is characterized by the progressive impairment of growth control mechanisms in which growth factors, oncogenes and tumor suppressor genes are involved. A general theme in carcinogenesis and tumorigenesis is the down regulation of the GJIC. Permeability of gap junctions in tumor cells using the dye-transfer assay is typically lower than GJIC in surrounding tissue. Further, the gating of gap junctions is known to be effected by tumor promoters, which decrease GJIC. Therefore, in one aspect, isopeptides according to the invention are used as medicaments for the treatment of cancer, alone, or in conjunction with traditional anti-cancer therapies.

Wound Healing

In a further aspect, isopeptides according to the invention are used to treat wounds and, in particular, to accelerate wound healing. Wound healing involves the interactions of many cell types, and intercellular communication mediated by gap junctions is considered to play an important role in the coordination of cellular metabolism during the growth and development of cells involved in tissue repair and regeneration (K. M. Abdullah, et al. (1999) Endocrine, 10 35-41; M. Saitoh, et al. (1997) Carcinogenesis, 18: 1319-1328; J. A. Goliger, et al. (1995) Mol. Biol. Cell, 6 1491-1501). Isopeptides may be administered to the site of a wound by topical administration using carriers well known in the art (e.g., ointments, creams, etc.) or may administered systemically, e.g., for treating wounds of internal tissues, such as in the treatment of chronic gastric ulcer lesions.

Ischemia

Additional functions in which endothelial gap-junctional intercellular communication has been implicated are the migratory behavior of endothelial cells after injury, angiogenesis, endothelial growth and senescence, and the coordination of vasomotor responses (G. J. Christ, et al. (2000) Brazi J Med Biol. Res., 33: 423-429). Therefore, in one aspect, a isopeptide according to the invention is used to enhance conducted vascular responses and to improve blood supply during conditions with increased metabolic demand (e.g., physical exercise, tachycardia), and during ischemia.

Gap junctions are also believed to provide the molecular link for co-ordinated long-range signaling among individual members of the glial compartment. Likewise, astrocytes are ideally suited for metabolic support of neurons since they are functionally polarized with one extremity touching the vascular bed and the other pole approximates neuronal parenchyma (R. Dermietzel (1998) Brain Res. Brain Res. Rev., 26: 176-183). Therefore, in one preferred embodiment, isopeptides according to the invention are administered to an individual in need to prevent ischemic damage in the brain by increasing the metabolic support between glia cells and neurons. Such individuals may include individuals with organic psychoses, which may present with signs such as depression, anxiety, learning and memory deficit, phobias, and hallucinations or individuals who have suffered a traumatic brain injury. Preferably, such isopeptides are selected or formulated so as to be available to the central nervous system (i.e., provided with or conjugated with carriers which facilitate transport across the blood-brain barrier).

Isopeptides according to the invention may also be used to accelerate repair after nerve injury or during grafting of immature cells (progenitor cells) into brain tissue, e.g., such as in individuals with neurotrauma, brain ischemia and chronic neurodegenerative diseases, such as Parkinson's disease or Huntington's disease (H. Aldskogius, et al. (1998) Prog. Neurobiol, 55: 1-26).

In specific embodiments, a isopeptide according to the present invention may, due to the effect on the intercellular gap junction channels, be used to treat and/or prevent cataract (D. Mackay, et al. (1999) Am J Hum. Genet, 64 1357-1364) treat and/or prevent vascularization of the cornea in disease states with poor nutrition of the cornea and increase the healing of corneal lesions (S. G. Spanakis, et al. (1998) Invest Ophthalmol. Vis. Sci., 39: 1320-1328) and/or prevent hypertension.

It should be obvious to those of skill in the art, that the isopeptides and pharmaceutical compositions according to the invention can be used to treat any condition or pathology associated with impaired (abnormal decreases or increases in) gap junctional communication. Preferably, one or more of the isopeptides or pharmaceutical compositions comprising the one or more isopeptides are administered to an individual in need thereof in a therapeutically effective amount. As used herein, “a therapeutically effective amount” is one which reduces symptoms of a given condition or pathology, and preferably which normalizes physiological responses in an individual with the condition or pathology. Reduction of symptoms or normalization of physiological responses can be determined using methods routine in the art and may vary with a given condition or pathology. In one aspect, a therapeutically effective amount of one or more isopeptides or pharmaceutical composition comprising the one or more isopeptides is an amount which restores a measurable physiological parameter to substantially the same value (preferably to within ±30%, more preferably to within ±20%, and still more preferably, to within 10% of the value) of the parameter in an individual without the condition or pathology.

Experimentals

The invention will now be further illustrated with reference to the following examples. It will be appreciated that what follows is by way of example only and that modifications to detail may be made while still falling within the scope of the invention.

### EXAMPLE 1

### Isopeptide Synthesis

A preferred general procedure is described below. However, more detailed descriptions of solid phase isopeptide syntheses are found in WO98/11125 hereby incorporated in its entirety.

a. General Isopeptide Synthesis

Apparatus and Synthetic Strategy

Isopeptides were synthesized batchwise in a polyethylene vessel equipped with a polypropylene filter for filtration using 9-fluorenylmethyloxycarbonyl (Fmoc) as N-α-amino protecting group and suitable common protection groups for side-chain functionalities.

Solvents

Solvent DMF (N,N-dimethylformamide, Riedel de-Häen, Germany) was purified by passing through a column packed with a strong cation exchange resin (Lewatit S 100 MB/H strong acid, Bayer AG Leverkusen, Germany) and analyzed for free amines prior to use by addition of 3,4-dihydro-3-hydroxy-4-oxo-1,2,3-benzotriazine (Dhbt-OH) giving rise to a yellow color (Dhbt-O-anion) if free amines are present. Solvent DCM (dichloromethane, analytical grade, Riedel de-Häen, Germany) was used directly without purification. Acetonitril (HPLC-grade, Lab-Scan, Dublin Ireland) was used directly without purification.

Amino Acids

Fmoc- and Boc-protected amino acids were purchased from Advanced ChemTech (ACT),

Bachem, NovaBiochem and Neosystem.

Benzoic acid and Benzyl Amine Derivatives

Benzoic acid and Benzyl amine derivatives were purchased from Aldrich and used without further purification.

Coupling Reagents

Coupling reagent diisopropylcarbodiimide (DIC) was purchased from (Riedel de-Häen, Germany), PyBop from Advanced ChemTech.

Linkers

(4-hydroxymethylphenoxy)acetic acid (HMPA), was purchased from Novabiochem, Switzerland; and was coupled to the resin as a preformed 1-hydroxybenzotriazole (HOBt) ester generated by means of DIC.

Solid Supports

Isopeptides synthesized according to the Fmoc-strategy on TentaGel S resins 0.22-0.31 mmol/g (TentaGel-S-NH₂; TentaGel S-Ram, Rapp polymere, Germany);

Catalysts and Other Reagents

Diisopropylethylamine (DIEA) was purchased from Aldrich, Germany, and ethylenediamine from Fluka, piperidine and pyridine from Riedel-de Häen, Frankfurt, Germany. 4-(N,N-dimethylamino)pyridine (DMAP) was purchased from Fluka, Switzerland and used as a catalyst in coupling reactions involving symmetrical anhydrides. Ethandithiol was purchased from Riedel-de Häen, Frankfurt, Germany. 3,4-dihydro-3-hydroxy-4-oxo-1,2,3-benzotriazine (Dhbt-OH), 1-hydroxybenzotriazole (HOBt) (HOAt) were obtained from Fluka, Switzerland.

Coupling Procedures

The first amino acid was coupled as a symmetrical anhydride in dmf generated from the appropriate n-α-protected amino acid and dic. The following amino acids were coupled as in situ generated hobt or hoat esters made from appropriate n-a-protected amino acids and hobt or hoat by means of dic in dmf. The acylations were checked by the ninhydrin test performed at 80° C. in order to prevent Fmoc deprotection during the test (B. D. Larsen, A. Holm, int.j pept.protein res. 1994, 43 1-9).

Deprotection of the N-α-amino protecting group (Fmoc).

Deprotection of the Fmoc group was performed by treatment with 20% piperidine in DMF (1×5 and 1×10 min.), followed by wash with DMF (5×15 ml, 5 min. each) until no yellow color could be detected after addition of Dhbt-OH to the drained DMF.

Deprotection of Allyl/Aloc

A solution of 3 eq. Pd(PPh₃)₄ dissolved in 15-20 ml CHCl₃, AcOH, NMM (37:2:1) was added to the peptid resin. The treatment was continued for three hours at room temperature accompanied by bubbling a stream of N₂ through the mixture.

Coupling of HOBt-Esters

3 eq. N-α-amino protected amino acid was dissolved in DMF together with 3 eq. HOBt and 3 eq. DIC and then added to the resin.

Preformed Symmetrical Anhydride

6 eq. N-α-amino protected amino acid was dissolved in DCM and cooled to 0° C. DIC (3 eq.) was added and the reaction continued for 10 min. The solvent was removed in vacuo and the remanence dissolved in DMF. The solution was immediately added to the resin followed by 0.1 eq. of DMAP.

Cleavage of Isopeptide from Resin With Acid

Isopeptides were cleaved from the resins by treatment with 95% triflouroacetic acid (TFA, Riedel-de Häen, Frankfurt, Germany)-water v/v or with 95% TFA and 5% ethandithiol v/v at r.t. for 2 h. The filtered resins were washed with 95% TFA-water and filtrates and washings evaporated under reduced pressure. The residue was washed with ether and freeze dried from acetic acid-water. The crude freeze dried product was analyzed by high-performance liquid chromatography (HPLC) and identified by electrospray ionisation mass spectrometry (ESMS).

Batchwise Isopeptide Synthesis on TentaGel Resin (PEG-PS)

TentaGel resin (1 g, 0.22-0.31 mmol/g) was placed in a polyethylene vessel equipped with a polypropylene filter for filtration. The resin was swelled in DMF (15 ml), and treated with 20% piperidine in DMF to secure the presence of non-protonated amino groups on the resin. The resin was drained and washed with DMF until no yellow color could be detected after addition of Dhbt-OH to the drained DMF. HMPA (3 eq.) was coupled as a preformed HOBt-ester as described above and the coupling was continued for 24 h. The resin was drained and washed with DMF (5×5 ml, 5 min each) and the acylation checked by the ninhydrin test. The first amino acid was coupled as a preformed symmetrical anhydride as described above. The following amino acids according to the sequence were coupled as preformed Fmoc-protected HOBt esters (3 eq.) as described above. The couplings were continued for 2 h, unless otherwise specified. The resin was drained and washed with DMF (5×15 ml, 5 min each) in order to remove excess reagent. All acylations were checked by the ninhydrin test performed at 80° C. After completed synthesis the isopeptide-resin was washed with DMF (3×15 ml, 5 min each), DCM (3×15 ml, 1 min each) and finally diethyl ether (3×15 ml, 1 min each) and dried in vacuo.

Preparative HPLC Conditions

Preparative chromatography was carried out using a VISION Workstation (PerSeptive Biosystem) equipped with AFC2000 automatic fraction collector/autosampler. VISION-3 software was used for instrument control and data acquisition. For preparative HPLC, different columns were used such as Kromasil (EKA Chemicals) KR100-10-C8 100 Å, C-8, 10 um; CER 2230, 250×50, 8 mm or a VYDAC 218TP101550, 300 Å, C-18, 10-15 um, 250×50 mm. The buffer system included: A: 0.1% TFA in MQV; B: 0.085% TFA, 10% MQV, 90% MeCN. Flow rates 35-40 ml/min. The preferred column temperature was 25° C. UV detection was performed at 215 nm and 280 nm. Suitable gradients were used for individual isopeptides.

Analytical HPLC Conditions

Gradient HPLC analysis was done using a Hewlett Packard HP 1100 HPLC system consisting of a HP 1100 Quaternary Pump, a HP 1100 Autosampler a HP 1100 Column Thermostat and HP 1100 Multiple Wavelength Detector. Hewlett Packard Chemstation for LC software (rev. A.06.01) was used for instrument control and data acquisition.

For analytical HPLC, different columns were used such as VYDAC 238TP5415, C-18, 5 um, 300 Å, or a Jupiter, Phenomenex 00F-4053-E0; 5 um C-18, 300 Å150×4.6 mm and others. The buffer system included: A: 0.1% TFA in MQV; B: 0.085% TFA, 10% MQV, 90% MeCN. Flow rates were 1 ml/min. The preferred column temperature was 40° C. UV detection: was performed at 215 nm. As above, suitable gradients were used for the individual isopeptides.

Mass Spectroscopy

The isopeptides were dissolved in super gradient methanol (Labscan, Dublin, Ireland), Milli-Q water (Millipore, Bedford, Mass.) and formic acid (Merck, Damstadt, Germany) (50:50:0.1 v/v/v) to give concentrations between 1 and 10 μg/ml. The isopeptide solutions (20 μl) were analysed in positive polarity mode by ESI-TOF-MS using a LCT mass spectrometer (Micromass, Manchester, UK) accuracy of ±0.1 m/z.

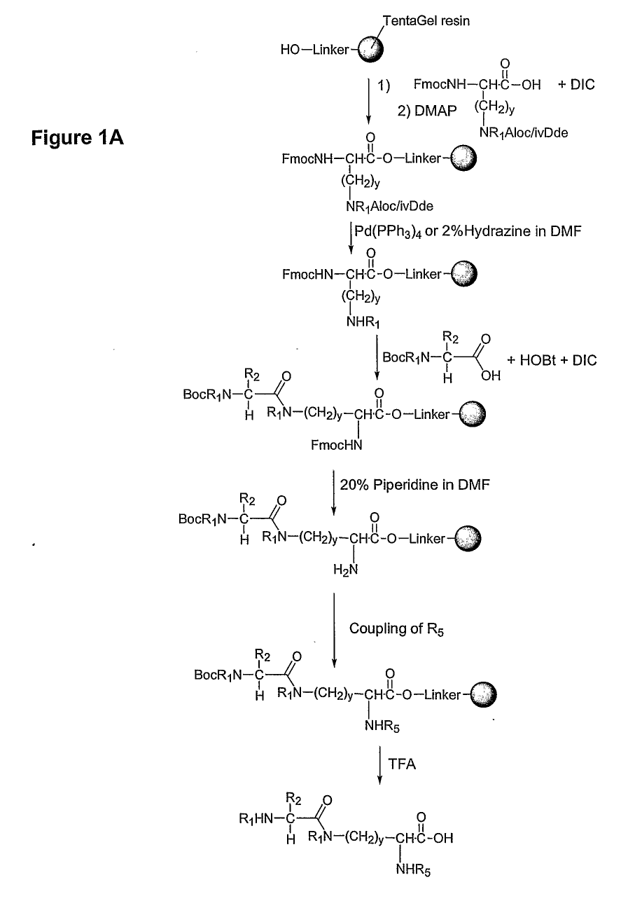

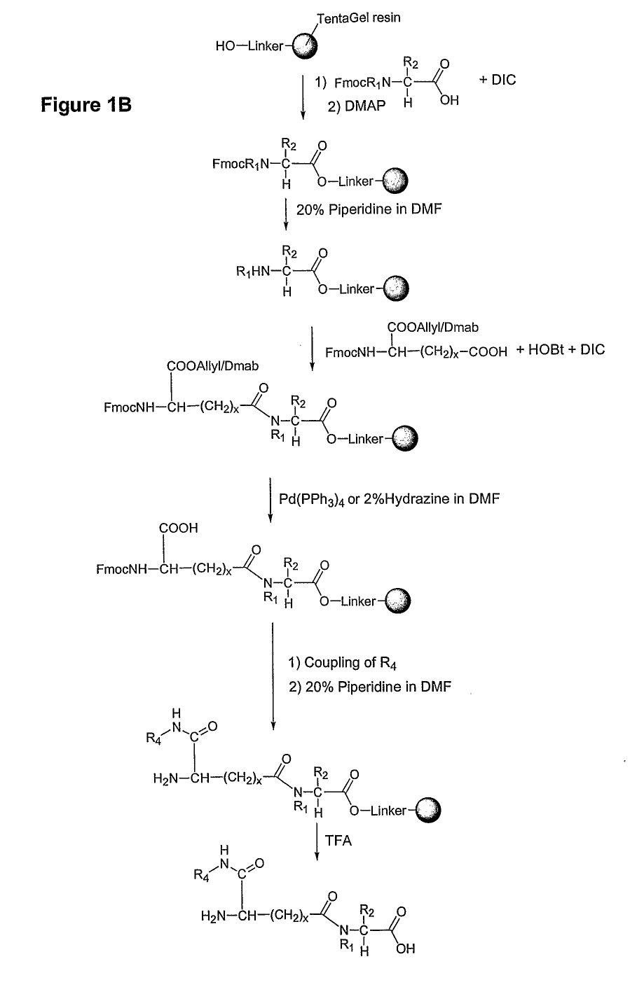

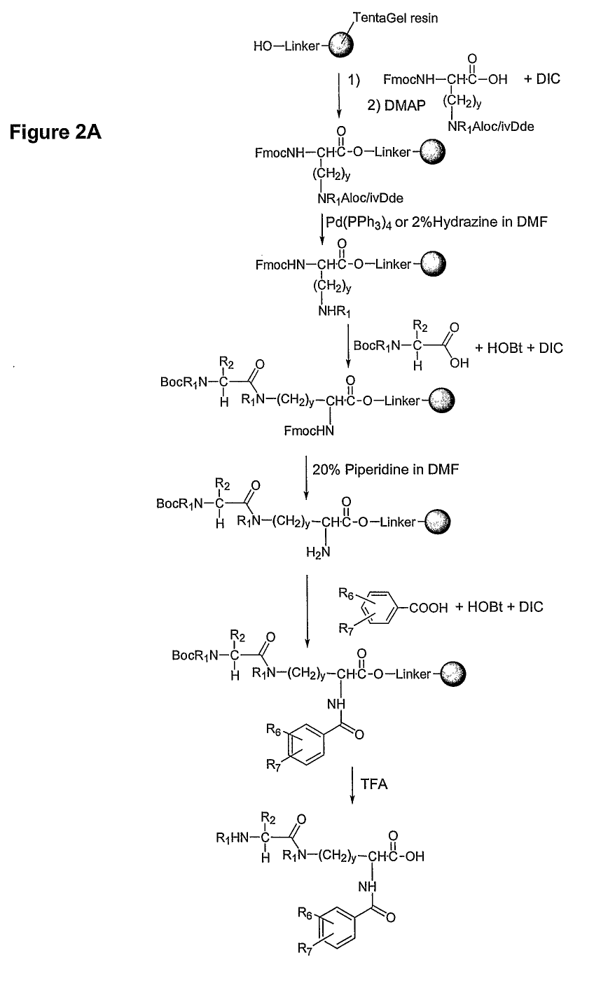

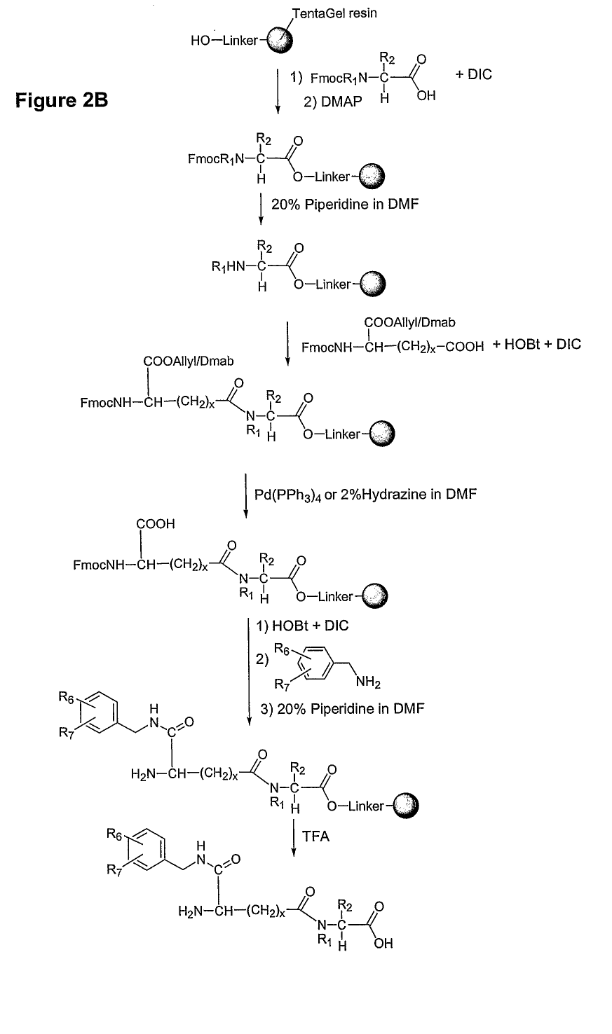

Exemplary synthesis schemes are shown in FIGS. 1A and 1B

b. Synthesis of Individual Isopeptides

### Synthesis of H-Gly-iso-Lys (4-nitrobenzoyl)-OH (Compound 1)

In this, and all subsequent syntheses described herein, dry TentaGel-S—NH₂ (0.23 mmol/g, 1 g) was placed in a polyethylene vessel equipped with a polypropylene filter for filtration and treated as described under “batchwise isopeptide synthesis on TentaGel resin.” Lysine was coupled as Fmoc-Lys(Aloc)-OH and Glycine as the Boc derivative. The Aloc protecting group was removed as described above. The Lysine was coupled to the solid support as the symmetrical anhydride followed by deprotection of the Aloc group. Glycine was then coupled to the ε-amino group of Lysine. The Fmoc group was then removed and subsequently 4-nitrobenzoic acid was coupled as an in situ generated HOBt ester by means of DIC in THF.

All couplings were continued for at least 2 hours. The acylations were checked by the ninhydrin test performed at 80° C. as earlier described. After completed synthesis the isopeptide-resin was washed with DMF (3×15 ml, 1 min each), DCM (3×15 ml, 1 min each), diethyl ether (3×15 ml, 1 min each) and dried in vacuo. The isopeptide was then cleaved from the resin as described above and freeze dried. This procedure was followed for all isopeptides exemplified further below.

After purification using preparative HPLC as described above, 40 mg isopeptide product was collected with a purity greater than 99%. The identity of the isopeptide was confirmed by ES-MS (found MH⁺352.07, calculated MH⁺352.24).

### Synthesis of H-Gly-iso-Lys (4-methoxybenzoyl)-OH (Compound 4)

Lysine was coupled as Fmoc-Lys(Aloc)-OH and Glycine as the Boc derivative. The Aloc protecting group was removed as described above. The Lysine was coupled to the solid support as the symmetrical anhydride followed by deprotection of the Aloc group. Glycine was then coupled to the ε-amino group of Lysine. The Fmoc group was then removed and subsequently 4-methoxybenzoic acid was coupled as an in situ generated HOBt ester by means of DIC in DMF.

After purification using preparative HPLC as described above, 25 mg isopeptide product was collected with a purity greater than 98%. The identity of the isopeptide was confirmed by ES-MS (found MH⁺337.16, calculated MH⁺337.27).

### Synthesis of H-Gly-iso-D-Lys (4-methoxybenzoyl)-OH (Compound 18)

Lysine was coupled as Fmoc-D-Lys(Aloc)-OH and Glycine as the Boc derivative. The Aloc protecting group was removed as described above. The Lysine was coupled to the solid support as the symmetrical anhydride followed by deprotection of the Aloc group. Glycine was then coupled to the ε-amino group of Lysine. The Fmoc group was then removed and subsequently 4-methoxybenzoic acid was coupled as an in situ generated HOBt ester by means of DIC in DMF.

After purification using preparative HPLC as described above, 35 mg isopeptide product was collected with a purity greater than 99%. The identity of the isopeptide was confirmed by ES-MS (found MH⁺337.13, calculated MH+337.21).

### Synthesis of H-Gly-iso-D-Lys (4-nitrobenzoyl)-OH (Compound 19)

Lysine was coupled as Fmoc-D-Lys(Aloc)-OH and Glycine as the Boc derivative. The Aloc protecting group was removed as described above. The Lysine was coupled to the solid support as the symmetrical anhydride followed by deprotection of the Aloc group. Glycine was then coupled to the ε-amino group of Lysine. The Fmoc group was then removed and subsequently 4-nitrobenzoic acid was coupled as an in situ generated HOBt ester by means of DIC in THF.

After purification using preparative HPLC as described above, 31 mg isopeptide product was collected with a purity greater than 99%. The identity of the isopeptide was confirmed by ES-MS (found MH⁺352.07, calculated MH⁺352.24).

### Synthesis of H-iso-Asn(NH((4-methoxybenzyl))-Ala-OH (Compound 57)

Alanine was coupled as Fmoc-Ala-OH and the Asparagine as the Boc-Asp-OFm derivative. The Fmoc protecting group was removed as described above. Alanine was coupled to the solid support as the symmetrical anhydride followed by deprotection of the Fmoc group. Aspartic acid was then coupled via the side chain carboxylic acid to the a-amino group of Alanine. The Fmoc group was then removed and subsequently 4-methoxybenzylamine was coupled to the a-carboxylic acid of the derivatized Asparagine by means of DIC and HOBt in DMF.

After purification using preparative HPLC as described above, 20 mg isopeptide product was collected with a purity greater than 98%. The identity of the isopeptide was confirmed by ES-MS (found MH⁺323.16, calculated MH⁺323.25).

### Synthesis of H-iso-Asn(NH((4-nitrobenzyl))-Ala-OH (Compound 58)

Alanine was coupled as Fmoc-Ala-OH and the Asparagine as the Boc-Asp-OFm derivative. The Fmoc protecting group was removed as described above. Alanine was coupled to the solid support as the symmetrical anhydride followed by deprotection of the Fmoc group. Aspartic acid was then coupled via side chain carboxylic acid to the a-amino group of Alanine.

The Fmoc group was then removed and subsequently 4-nitrobenzylamine was coupled to the α-carboxylic acid of the derivatized Asparagine by means of DIC and HOBt in DMF.

After purification using preparative HPLC as described above, 56 mg isopeptide product was collected with a purity greater than 98%. The identity of the isopeptide was confirmed by ES-MS (found MH⁺338.12, calculated MH⁺338.22).

### Synthesis of H-iso-Asn(NH((4-methoxybenzyl))-D-Ala-OH (Compound 72)

Alanine was coupled as Fmoc-D-Ala-OH and the Asparagine as the Boc-Asp-OFm derivative. The Fmoc protecting group was removed as described above. Alanine was coupled to the solid support as the symmetrical anhydride followed by deprotection of the Fmoc group. Aspartic acid was then coupled via side chain carboxylic acid to the α-amino group of Alanine. The Fmoc group was then removed and subsequently 4-methoxybenzylamine was coupled to the α-carboxylic acid of the derivatized Asparagine by means of DIC and HOBt in DMF.

After purification using preparative HPLC as described above, 16 mg isopeptide product was collected with a purity greater than 99%. The identity of the isopeptide was confirmed by ES-MS (found MH⁺323.16, calculated MH⁺323.24).

### Synthesis of H-iso-Asn(NH((4-nitrobenzyl))-D-Ala-OH (Compound 73)

Alanine was coupled as Fmoc-D-Ala-OH and the Asparagine as the Boc-Asp-OFm derivative. The Fmoc protecting group was removed as described above. Alanine was coupled to the solid support as the symmetrical anhydride followed by deprotection of the Fmoc group. Aspartic acid was then coupled via side chain carboxylic acid to the α-amino group of Alanine. The Fmoc group was then removed and subsequently 4-nitrobenzylamine was coupled to the α-carboxylic acid of the derivatized Asparagine by means of DIC and HOBt in DMF.

After purification using preparative HPLC as described above, 31 mg isopeptide product was collected with a purity greater than 99%. The identity of the isopeptide was confirmed by ES-MS (found MH⁺338.12, calculated MH⁺338.22).

### Synthesis of H-iso-Gln(NH((4-methoxybenzyl))-Ala-OH (Compound 101)

Alanine was coupled as Fmoc-Ala-OH and the Glutamine as the Boc-Glu-OFm derivative. The Fmoc protecting group was removed as described above. Alanine was coupled to the solid support as the symmetrical anhydride followed by deprotection of the Fmoc group. Glutamic acid was then coupled via the side chain carboxylic acid to the α-amino group of Alanine. The Fmoc group was then removed and subsequently 4-methoxybenzylamine was coupled to the α-carboxylic acid of the derivatized Glutamine by means of DIC and HOBt in DMF.

After purification using preparative HPLC as described above, 27 mg isopeptide product was collected with a purity greater than 99%. The identity of the isopeptide was confirmed by ES-MS (found MH⁺337.13, calculated MH⁺337.25).

### Synthesis of H-iso-Gln(NH((4-nitrobenzyl))-Ala-OH (Compound 102)

Alanine was coupled as Fmoc-Ala-OH and the Glutamine as the Boc-Glu-OFm derivative. The Fmoc protecting group was removed as described above. Alanine was coupled to the solid support as the symmetrical anhydride followed by deprotection of the Fmoc group. Glutamic acid was then coupled via the side chain carboxylic acid to the α-amino group of Alanine. The Fmoc group was then removed and subsequently 4-nitrobenzylamine was coupled to the α-carboxylic acid of the derivatized Glutamine by means of DIC and HOBt in THF.

After purification using preparative HPLC as described above, 22 mg isopeptide product was collected with a purity greater than 98%. The identity of the isopeptide was confirmed by ES-MS (found MH⁺352.15, calculated MH⁺352.2).

### Synthesis of H-iso-Gln(NH((4-methylbenzyl))-Ala-OH (Compound 107)

Alanine was coupled as Fmoc-Ala-OH and the Glutamine as the Boc-Glu-OFm derivative. The Fmoc protecting group was removed as described above. Alanine was coupled to the solid support as the symmetrical anhydride followed by deprotection of the Fmoc group. Glutamic acid was then coupled via the side chain carboxylic acid to the α-amino group of Alanine. The Fmoc group was then removed and subsequently 4-methylbenzylamine was coupled to the α-carboxylic acid of the derivatized Glutamine by means of DIC and HOBt in DMF.

After purification using preparative HPLC as described above, the isopeptide product was collected and the identity of the isopeptide was confirmed by ES-MS.

### Synthesis of H-iso-Gln(NH((4-methoxybenzyl))-D-Ala-OH (Compound 116)

Alanine was coupled as Fmoc-D-Ala-OH and the Glutamine as the Boc-Glu-OFm derivative. The Fmoc protecting group was removed as described above. Alanine was coupled to the solid support as the symmetrical anhydride followed by deprotection of the Fmoc group. Glutamic acid was then coupled via the side chain carboxylic acid to the α-amino group of Alanine. The Fmoc group was then removed and subsequently 4-methoxybenzylamine was coupled to the α-carboxylic acid of the derivatized Glutamine by means of DIC and HOBt in DMF.

After purification using preparative HPLC as described above, 11 mg isopeptide product was collected with a purity greater than 98%. The identity of the isopeptide was confirmed by ES-MS (found MH⁺337.22, calculated MH⁺337.28).

### Synthesis of H-iso-Gln(NH((4-nitrobenzyl))-D-Ala-OH (Compound 117)

Alanine was coupled as Fmoc-D-Ala-OH and the Glutamine as the Boc-Glu-OFm derivative. The Fmoc protecting group was removed as described above. Alanine was coupled to the solid support as the symmetrical anhydride followed by deprotection of the Fmoc group. Glutamic acid was then coupled via side chain carboxylic acid to the α-amino group of Alanine. The Fmoc group was then removed and subsequently 4-nitrobenzylamine was coupled to the α-carboxylic acid of the derivatized Glutamine by means of DIC and HOBt in DMF.

After purification using preparative HPLC as described above, 24 mg isopeptide product was collected with a purity greater than 96%. The identity of the isopeptide was confirmed by ES-MS (found MH⁺352.12, calculated MH⁺352.22).

### EXAMPLE 2

### Effect of Isopeptides on Calcium Induced Arrhythmias

The anti-arrhythmic effect of isopeptides was tested in a model of calcium-induced arrhythmias according to the model of Lynch et al., J. Cardiovasc. Pharmacol. (1981), 3: 49-60. Male NMRI mice (25-30 grams; Bomholdtgaard, L I. Skendsved, Denmark) were anesthetized with a neurolept anesthetic combination (Hynorm® (fentanyl citrate 0.315 mg/ml and fuanisone 10 mg/ml) and midazolam at 5 mg/ml. Commercial solutions of Hynorm® and midazolam were diluted 1:1 in distilled water, and one part Hynorm® was mixed with one part diluted midazolam. Anesthesia is induced by s.c. administration of this solution in a dose of 50-75 ul/10 gram mouse.

An intravenous cannula was inserted into the tail vein. The lead II ECG signal was recorded continuously by positioning of stainless steel ECG electrodes on the right forelimb and on the left hind limb. The ground electrode was placed on the right hind limb. The signal was amplified ×5.000-10.000) and filtered (0.1-150 Hz) via a Hugo Sachs Electronic model 689 ECG module. The analogue signal was digitized via a 12-bit data acquisition board (Data Translation model DT321) and sampled at 1000 Hz using the Notocord HEM 3.1 software for Windows NT. After a 10-min equilibration period, the test sample of isopeptide was injected into the tail vein at a dose of 1 nmol/kg and three minutes later intravenous infusion of CaCl₂ (30 mg/ml, 0.1 ml/min˜100 mg/kg/min, calcium chloride-2-hydrate, Riedel-de Haen, Germany) was started.

Mice pre-treated with vehicle (phosphate buffered saline with 0.1% bovine albumin) were tested on all days as a measure for control level in untreated animal. Injection volume was 100 ul in all experiments. The time lag to onset of arrhythmias was determined as the time from the start of CaCl₂ infusion until the first event of conduction block (defined as intermittent failure of the SA or AV conduction characterized by delayed P-wave activation (SA block) or by a P-wave without he concomitant QRS complex (AV block).

Results:

The % response of the tested peptide compounds are given below in Table 2. The response is estimated according to (tₐᵣᵣ(testcompound)−tₐᵣᵣ(vehicle))×100/tₐᵣᵣ (vehicle).

TABLE 2

| Com- | | % re- | |
|---|---|---|---|
| pound | Name | sponse | SEM+/− |
| 19 | H-Gly-iso-D-Lys(4-nitrobenzoyl)- | 33 | 6 |
| | OH | | |
| 18 | H-Gly-iso-D-Lys(4-methoxybenzoyl)- | 16 | 12 |
| | OH | | |
| 101 | H-iso-Gln(NH(4-methoxybenzyl))- | 68 | 10 |
| | Ala-OH | | |
| 116 | H-iso-Gln(NH(4-methoxybenzyl)-D- | 25 | 16 |
| | Ala-OH | | |

Conclusion

The peptides of the invention showed an antiarrhythmic effect.

### DEFINITIONS

Unless specified otherwise, the following definitions are provided for specific terms, which are used in the following written description.

Throughout the description and claims the three-letter code for natural amino acids is used as well as generally accepted three letter codes for other a-amino acids, such as Sarcosin (Sar). Where the L or D form has not been specified, it is to be understood that the amino acid in question can be either the L or D form.

By “functional analogs or derivatives or modified forms” of a isopeptide is meant any chemical entity or compound which has a structural conformation and/or binding properties that are sufficiently similar to the endogenous AAP or a functional analog thereof (e.g., such as AAP10 or HP5) or which binds to a receptor bound by AAP to provide one or more of the beneficial effects of maintaining or normalizing gap junction function (i.e., enhancing when gap junctional communication is impaired or inhibiting when gap junctional communication is overstimulated or uncontrolled). Preferably, such analogs or derivatives are also able to bind to the isopeptide carrier hPepT1 or a structural analogue thereof. As used throughout the specification and claims, the term “isopeptide” is inclusive of an isopeptide, or a functional analogue or derivative of such an isopeptide as defined above. The term “functional analog of a isopeptide” is further inclusive of peptidomimetics or peptoids.

The term “isopeptide mimetic” refers to compounds of both isopeptide and non-isopeptide nature. The objective behind the creation of peptidomimetics is to create scaffolds, which can replace the isopeptide backbone. It is assumed that the secondary amide bonds in isopeptides are responsible for instability and possibly poor isopeptide transport properties across cell membranes. Proper placement of amino acid side chains with appropriate trajectories is viewed as the key design tactic in isopeptide peptidomimetics to achieve biological activity. The backbone modifications include reduced amide bonds and alkylated amide bonds and the use of isosteric bonds such as thioamide bonds, CH₂—CH₂, CH═CH. The term “peptoid” refers to compounds that may be characterised by topological similarity between the structural formula of the peptoid and the parent isopeptide. Thus, a peptoid may be a compound consisting of isopeptide-like chains of amino acids bearing side chains on the backbone nitrogen atom rather than on the alpha-carbon as in true isopeptides. Petidomimetics and peptoids may comprise amino acid units having modified side chains, such as NaI, Dab, and Dapa, or they may comprise D-amino acids. The various modifications of isopeptide and peptidomimetic structure described by El Tayar, N et al. (Amino Acids (1995) 8: 125-139) are included in the definitions herein.

The term “halogen” refers to F, Cl, Br, and I, where F and I are preferred.

The term “alkyl” refers to univalent groups derived from alkanes by removal of a hydrogen atom from any carbon atom: CₙH₂ₙ₊₁—. The groups derived by removal of a hydrogen atom from a terminal carbon atom of unbranched alkanes form a subclass of normal alkyl (n-alkyl) groups: H[CH₂]ₙ—. The groups RCH₂—, R₂CH— (R not equal to H), and R₃C— (R not equal to H) are primary, secondary and tertiary alkyl groups respectively. C(1-22)alkyl refers to any alkyl group having from 1 to 22 carbon atoms and includes C(1-6)alkyl, such as methyl, ethyl, propyl, iso-propyl, butyl, pentyl and hexyl and all possible isomers thereof.

By the phrase “lower alkyl” is meant a linear or branched alkyl having less than about 6 carbon atoms, preferably methyl, ethyl, propyl, or butyl.

The term “alkenyl” refers to a straight or branched or cyclic hydrocarbon group containing one or more carbon-carbon double bonds. C(2-22)alkenyl refers to any alkenyl group having from 1 to 22 carbon atoms and includes C(2-6)alkenyl, vinyl, allyl, 1-butenyl, etc.

The term “aralkyl” refers to aryl C(1-22)alkyl, and the term “aryl” throughout this specification means phenyl or naphthyl.

By the phrase “hydrophobic group” is meant an optionally substituted aromatic carbon ring, preferably a 6 or 12-membered aromatic carbon ring. By the phrase “optionally substituted” is meant substitution of the 6 or 12-membered aromatic carbon ring with at least one of a lower alkyl, alkoxy, hydroxyl, carboxy, amine, thiol, hydrazide, amide, halide, hydroxyl, ether, amine, nitrile, imine, nitro, sulfide, sulfoxide, sulfone, thiol, aldehyde, keto, carboxy, ester, an amide group; including seleno and thio derivatives thereof. Also included in the definition of “optionally substituted” are are sulfide, sulfoxide, sulfone, and thiol derivates with or without a seleno group. In embodiments in which the aromatic carbon ring is substituted such substitutions will typically number less than about 10 substitutions, more preferably about 1 to to 5 of same with about 1 or 2 substitutions being preferred for many invention applications. Preferred alkoxy groups include methoxy, ethoxy, and propoxy. Illustrative hydrophobic groups include unsubstituted benzyl, phenyl, and napthyl.

By the phrase “hydrogen bond group” is meant a donor or acceptor of a (non-covalent) hydrogen bond. In embodiments in which the hydrogen bond group is the donor, it will typically include at least one electronegative atom bound to a hydrogen atom. Examples of such electronegative atoms include, but are not limited to, nitrogen, oxygen, halide (eg., Cl, F, Br, etc), and sulfur. Illustrative hydrogen bond donors include hydroxyl, amine, thiol, hydrazide and amide. In embodiments in which the first hydrogen bond group is an acceptor, it will preferably include at least one electronegative atom such as those mentioned above in which the atom includes a non-bonded electron pair. Such pairs are often referred to as “lone pairs”. Examples of preferred hydrogen bond acceptors include, but are not limited to, halide, hydroxyl, ether, amine, nitrile, imine, nitro, aldehyde, keto, carboxy, ester, amide group; and seleno derivatives thereof. Also envisioned are suitable sulfide, sulfoxide, sulfone, and thiol hydrogen bond acceptors including seleno derivatives thereof.

The terms “intercellular communication modulator”, “gap junction facilitator”, “compound that facilitates gap junction communication” and “gap junction opener”, etc., all refer to a compound that facilitates, or maintains, or normalizes, GJIC, irrespective of the particular mechanism behind this action. More specifically, the term “gap junction opener” may refer to a substance which normalizes (i.e., increases) the exchange of molecules that are able to pass through gap junctions between extracellular and intracellular spaces and/or which can normalize increase GJIC.

The term “agonist” “refers to an isopeptide that can interact with a tissue, cell or cell fraction which is the target of an AAP, AAP10, HP5 isopeptide, or functional analog thereof, to cause substantially the same physiological responses in the tissue, cell or cell fraction as the AAP, AAP10, HP5 isopeptide, or functional analog thereof. In one aspect, the physiological response is one or more of: contraction, relaxation, secretion, enzyme activation, etc. Preferably, the isopeptide binds to the tissue, cell or cell fraction. In one aspect, the isopeptide binds to a receptor on the tissue, cell, or cell fraction, which binds to AAP, AAP10, HP5, or a functional analog thereof.

An “antiarrhythmic isopeptide agonist” as used herein is an isopeptide, which comprises an antiarrhythmic activity, which is substantially the same, or greater than, the antiarrhythmic activity of an AAP, AAP10, HP5 isopeptide or functional analog thereof. “Greater than” refers to an antiarrhythmic activity, which is observed at lower concentrations of isopeptide or in shorter periods of time compared to the antiarrhythmic activity of an AAP, AAP10, HP5 isopeptide or functional analog thereof.

The term “antagonist” refers to a isopeptide which inhibits or antagonizes one or more physiological responses observed in a tissue, cell or cell fraction after contacting the tissue, cell, or cell fraction with AAP, AAP10, HP5 isopeptide, or a functional analog thereof. In one aspect, the physiological response is one or more of: contraction, relaxation, secretion, enzyme activation, etc. Preferably, the isopeptide binds to the tissue, cell or cell fraction. In one aspect, the isopeptide binds to a receptor on the tissue, cell, or cell fraction which binds to AAP, AAP10, HP5, or a functional analog thereof and/or which inhibits binding of one or more of AAP, AAP10, HP5, a functional analog thereof, to the receptor.

As used herein, “normalize” refers to a change in a physiological response such that the response becomes insignificantly different from one observed in a normal patient. Thus, normalization may involve an increase or decrease in the response depending on the pathology involved.

The “IC50” of a isopeptide according to the invention refers to the concentration of a isopeptide that is required for 50% inhibition of a response or activity mediated by an antiarrhythmic isopeptide such as AAP, AAP10, HP5 or a functional analog thereof. In one-aspect, a isopeptide which is an antagonist of AAP, AAP10, HP5 or a functional analog thereof, is a isopeptide which has an IC50 of less than about 10⁻⁶M, and preferably, less than about 10⁻⁸ M.

The “EC50” of a isopeptide according to the invention refers to the plasma concentration/AUC of isopeptide required for obtaining 50% of a maximum effect observed for an MP, AAP10, HP5 isopeptide or a functional analog thereof. In one aspect, a isopeptide which is an agonist of AAP, AAP10, HP5 or a functional analog thereof, is a isopeptide which has an EC50 of less than about 10⁻⁶M, and preferably, less than about, 10⁻⁸ M.

As used herein, “oral availability” refers to the rate and extent of absorption of an orally administered drug into the blood stream.

Abbreviations used in the instant application are defined further below.

| | | | |
|---|---|---|---|
| Methyl (Me) | —CH₃ | Methoxy (MeO) | —OCH₃ |
| Ethyl (Et) | —CH₂CH₃ | Ethoxy (EtO) | —OCH₂CH₃ |
| n-Butyl (n-Bu) | —CH₂CH₂CH₂CH₃ | n-Butoxy (n-BuO) | —OCH₂CH₂CH₂CH₃ |
| n-Hexyl (n-Hex) | —CH₂CH₂CH₂CH₂CH₂CH₃ | n-Hexyloxy (n-HexO) | —OCH₂CH₂CH₂CH₂CH₂CH₃ |
| n-Octyl (n-Oct) | —CH₂CH₂CH₂CH₂CH₂CH₂CH₂CH₃ | n-Octyloxy (n-OctO) | —OCH₂CH₂CH₂CH₂CH₂CH₂CH₂CH₃ |
| t-Butyl (t-Bu) | 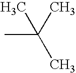 | t-Butoxy (t-BuO) | 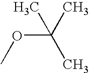 |
| cyclo-hexyl (c-Hex) | 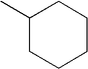 | cyclo-hexyloxy (o-HexO) | 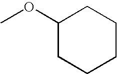 |
| Phenyl (Ph) | 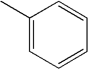 | Phenoxy (PhO) | 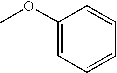 |
| Benzyl (Bzl) | 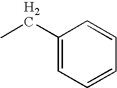 | Benzyloxy (BzlO) | 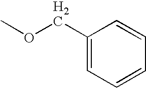 |

All references cited herein are incorporated by reference in their entireties herein.

Variations, modifications, and other implementations of what is described herein will occur to those of ordinary skill in the art without departing from the spirit and scope of the invention and the claims herein.

1. An isopeptide represented by the general formula (I): 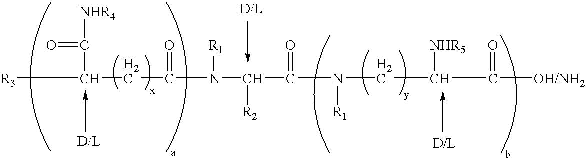
where, if a is 1 then b is 0;
if a is 0 then b is 1;
x and y independently are 1-7;
R₁ is H or CH₃;
R₂ is the side chain of an amino acid selected from the group alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine;
R₃ is selected from the group consisting of: H, NH₂, NHR, NR₂, ⁺NR₃, OH, SH, RO, RS, RSO, RSO₂, COR, CSR, COOH, COOR, CONH₂, CONHR, CONR₂, OCOR, and SCOR, wherein R=alkyl, alkenyl, aryl, aralkyl, or cycloalkyl; and
R₄ and R₅ are independently a hydrophobic group.

2. The isopeptide according to claim 1, wherein R₁ is H.

3. The isopeptide according to claim 1, wherein R₂ is the side chain of an amino acid selected from the group consisting of glycine and alanine.

4. The isopeptide according to claim 1, wherein R₃ is H or NH₂.

5. The isopeptide according to claim 1, wherein R₄ and R₅ independently comprise an aromatic carbon ring.

6. The isopeptide according to claim 5, wherein the aromatic ring comprises a 6- or 12 membered ring or a substituted form thereof.

7. The isopeptide according to claim 6, wherein the ring is substituted with at least one of: a lower alkyl, alkoxy, hydroxyl, carboxy, amine, thiol, hydrazide, amide, halide, hydroxyl, ether, amine, nitrile, imine, nitro, sulfide, sulfoxide, sulfone, thiol, aldehyde, keto, carboxy, ester, an amide group; a seleno group, a thio group and derivatives thereof.

8. The isopeptide according to claim 6, wherein the aromatic ring is substituted with at least one of: a lower alkyl, alkoxy, halide, nitrile and nitro group.

9. The isopeptide according to claim 6, wherein the ring is substituted with at least one of: an alkoxy and nitro group.

10. The isopeptide according to claim 6, wherein the ring comprises about 1 to 5 substitutions.

11. The isopeptide according to claim 6, wherein the ring comprises about 1 to 2 substitutions.

12-67. (canceled)

68. The isopeptide according to claim 6, wherein the 6-membered aromatic carbon ring comprises a substituent at the 4-position.

69. The isopeptide according to claim 68, wherein the substituent is selected from the group consisting of a methyl, ethyl, t-butyl, c-hexyl, phenyl, n-butyl, n-hexyl, n-octyl, ethoxy, t-butoxy, phenoxy, butoxy, benzyloxy, n-hexyloxy, and n-octyloxy group.

70. The isopeptide according to claim 5, wherein the aromatic carbon ring is selected from the group consisting of a benzyl, phenyl, and napthyl group.

71. The isopeptide according to claim 70, wherein the aromatic carbon ring is a benzyl group.

72. The isopeptide according to claim 1, wherein R₁ is H; R₂ is the side chain of the amino acid glycine or alanine; R₃ is H or NH₂; and R4 and R₅ comprise a benzyl group substituted with at least one of a nitro or methoxy group.

73. The isopeptide according to claim 1, wherein the isopeptide comprises a free N-terminal, a free C-terminal, or both a free N— and C-terminal.

74. The isopeptide according to claim 1, wherein the isopeptide has a property selected from the group consisting of binding to an hPepT1 transporter or a biologically active fragment thereof; having a half-life in an in vitro plasma stability assay of more than about 30 minutes; having a half-life in an in vitro plasma stability assay of more than about 48 hours; binding to a tissue, cell, or cell fraction that is a site of action for an antiarrhythmic peptide; modulating the function of the tissue, cell, or cell fraction; antagonizing the function of the antiarrhythmic peptide; agonizing the function of the antiarrhythmic peptide; modulating a receptor of the antiarrhythmic peptide; and increasing the time to an AV block in a standard calcium-induced arrhythmia assay.

75. The isopeptide according to claim 1, wherein the isopeptide is selected from the group consisting of the isopeptides shown in Table 1 and Table 2.

76. A pharmaceutical composition comprising an isopeptide of claim 1 and a pharmaceutical carrier.

77. The pharmaceutical composition according to claim 76, wherein the composition is parenterally administrable or is orally administrable.

78. An isopeptide represented by the general formula II: 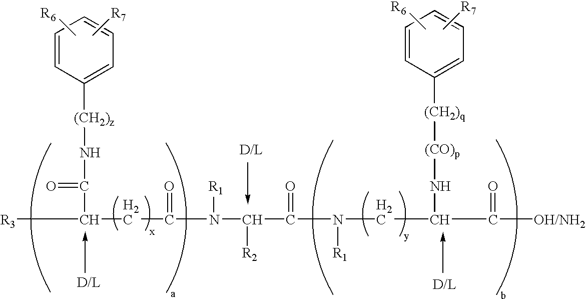
where, if a is 1 then b is 0;
if a is 0 then b is 1;
x and y independently are 1-7;
z is 1-6;
q is 0-6;
p is 0-1;
R₁ is H or CH₃;
R₂ is the side chain of an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine;
R₃ is selected from the group consisting of: H, NH₂, NHR, NR₂, ⁺NR₃, OH, SH, RO, RS, RSO, RSO₂, COR, CSR, COOH, COOR, CONH₂, CONHR, CONR₂, OCOR, and SCOR, wherein R=alkyl, alkenyl, aryl, aralkyl, or cycloalkyl; and
R₆ and R₇ are independently selected from the group consisting of H, alkyl, alkenyl, aryl, aralkyl, halogen, CN, NO₂, alkoxy, aryloxy, aralkyloxy, thioalkoxy, thioaryloxy, thioaralkyloxy, +S(CH₃)₂, SO₃H, SO₂R, NH₂, NHR, NR₂, ⁺NR₃, OH, SH, COOH, COOR, CONH₂, CONHR, CONR₂, CH₂OH,. NCO, NCOR, NHOH, NHNH₂, NHNRH, CH₂OCOR, CH₂OCSR, COR, CSR, CSOR, CF₃, and CCl₃, wherein R is alkyl, alkenyl, aryl, aralkyl, or cycloalkyl.

79. The isopeptide according to claim 78, wherein R₁ is H.

80. The isopeptide according to claim 78, wherein R₂ is the side chain of an amino acid selected from the group consisting of glycine and alanine.

81. The isopeptide according to claim 78, wherein R₃ is H or NH₂.

82. The isopeptide according to claim 78, wherein R₆ and R₇ are independently selected from the group consisting of H, alkyl, halogen, CN, NO₂, alkoxy and CF₃.

83. The isopeptide according to claim 82, wherein R₆ and R₇ are independently selected from the group consisting of H, NO₂, and alkoxy.

84. The isopeptide according to claim 78, wherein R₁ is H; R₂ is the side chain of the amino acid glycine or alanine; R₃ is H or NH₂; R₆ and R₇ are independently selected from the group consisting of H, NO₂, and methoxy.

85. A method for modulating gap junctional communication in a population of cells comprising administering an effective amount of an isopeptide of claim 1 to the population of cells, thereby modulating gap junctional communication between the cells.

86. A method of preventing and/or treating a pathological condition involving impaired gap junctional communication comprising administering to an individual in need thereof a therapeutically effective amount of an isopeptide of claim 1.

87. The method according to claim 86, wherein administration is parenteral or oral.

88. The method according to claim 86, wherein the pathological condition is selected from the group consisting of a cardiovascular disease; inflammation of airway epithelium; a disorder of alveolar tissue; bladder incontinence; impaired hearing; an endothelial lesion; diabetic retinopathy; diabetic neuropathy; a central nervous system disorder; ischemia of the central nervous system, spinal cord, brain, or brain stem; a dental tissue disorder; kidney disease; failure of bone marrow transplantation; wound; erectile dysfunction; neuropathic pain; subchronic inflammation; chronic inflammation; cancer; transplantation failure; and a condition caused by an excess of reactive oxygen species, free radicals, or nitric oxide.
